(19) United States
(12) Patent Application Publication
(10) Pub. No.: US 2025/0019758 A1
(43) Pub. Date: Jan. 16, 2025

(54) CONTROLLED POLYNUCLEOTIDE TRANSLOCATION IN NANOPORE SEQUENCING

(71) Applicant: ILLUMINA, INC., San Diego, CA (US)

(72) Inventors: Abdul Sadeer Abd Salam, Singapore (SG); Xiangyuan Yang, Singapore (SG); Hassan Zakiruddin Bohra, Singapore (SG); Yin Nah Teo, Singapore (SG); Tsz Ying Yuen, Singapore (SG); Daniel Hartoyo Lukamto, Singapore (SG); Erin Garcia, San Diego, CA (US); Jeffrey G. Mandell, San Diego, CA (US)

(21) Appl. No.: 18/650,800

(22) Filed: Apr. 30, 2024

Related U.S. Application Data
(60) Provisional application No. 63/499,960, filed on May 3, 2023.

(51) Int. Cl. C12Q 1/6869; C12Q 1/6806

(52) U.S. Cl. CPC C12Q 1/6869; C12Q 1/6806

(57) ABSTRACT

Provided herein are methods for modification-based controlled polynucleotide translocation in nanopores for sequencing, modified nucleotides, and kits and systems for performing the disclosed methods. In some embodiments, modifications can be used to control polynucleotide translocation by modifying nucleotides on a strand of polynucleotide to carry a modification, where the modifications can arrest or slow translocation when encountering the nanopore. In some embodiments, application of a voltage can move one nucleotide and its attached modification through the nanopore at a time.

### INCORPORATION BY REFERENCE TO RELATED APPLICATION

Any and all applications for which a foreign or domestic priority claim is identified in the Application Data Sheet as filed with the present application are hereby incorporated by reference under 37 CFR 1.57.

### REFERENCE TO SEQUENCE LISTING

The present application is being filed along with a replacement Sequence Listing in electronic format. The Sequence Listing is provided as a file entitled “ILLINC.613AReplacementSequenceListing.xml” which is 35, 062 bytes in size and was created on Sep. 16, 2024. The information in the electronic format of the Sequence Listing is incorporated herein by reference in its entirety.

### BACKGROUND

Some polynucleotide sequencing techniques involve performing a large number of controlled reactions on support surfaces or within predefined reaction chambers. The controlled reactions may then be observed or detected, and subsequent analysis may help identify properties of the polynucleotide involved in the reaction. Examples of such sequencing techniques include next-generation sequencing or massive parallel sequencing involving sequencing-by-ligation, sequencing-by-synthesis, reversible terminator chemistry, or pyrosequencing approaches.

Some polynucleotide sequencing techniques utilize a nanopore, which can provide a path for an ionic electrical current. For example, as the polynucleotide traverses through the nanopore, it influences the electrical current through the nanopore. Each passing nucleotide, or series of nucleotides, that passes through the nanopore yields a characteristic electrical current. These characteristic electrical currents of the traversing polynucleotide can be recorded to determine the sequence of the polynucleotide.

### SUMMARY

Provided in examples herein are methods for sequencing biopolymers, particularly polynucleotides, and systems and kits for performing the methods.

The systems, devices, kits, and methods disclosed herein each have several aspects, no single one of which is solely responsible for their desirable attributes. Without limiting the scope of the claims, some prominent features will now be discussed briefly. Numerous other examples are also contemplated, including examples that have fewer, additional, and/or different components, steps, features, objects, benefits, and advantages. The components, aspects, and steps may also be arranged and ordered differently. After considering this discussion, and particularly after reading the section entitled “Detailed Description,” one will understand how the features of the devices and methods disclosed herein provide advantages over other known devices and methods.

In one aspect, provided herein is a method for determining a sequence of a target polynucleotide in a nanopore-based sequencing system, the method comprises providing a target polynucleotide comprising nucleotides, wherein each nucleotide is attached to a modification configured to arrest the target polynucleotide relative to a nanopore. In one aspect, the modification comprises an arresting construct. In some embodiments, the modification further comprises a cyclic loop, wherein the arresting construct is attached to the cyclic loop. In some embodiments, the cyclic loop further comprises a reporter element that encodes the nucleotide. In some embodiments, the cyclic loop further comprises a spacer. The method may further comprise, applying a driving voltage to translocate one or more portions of the target polynucleotide through a nanopore, measuring a current of the nanopore continuously during translocation, and identifying the sequence of the target polynucleotide by correlating the measured current to an identity of one or more nucleotides.

The first reporter clement may comprise one or multiple nucleotides in addition to other moieties, e.g., modifications attached to nucleotides. The first electrical response may be an ionic current through the nanopore. The modifications may be covalently linked to the nucleotides. The modifications may interact with the nanopore non-covalently. In some embodiments, the first electrical response depends on the modification attached to nucleotides in the first reporter clement.

The method may further comprise applying a voltage for a duration of time that is sufficient to translocate the first reporter element through the nanopore. The method may further comprise, during a second arresting event, applying the reading voltage across the nanopore to identify a second reporter element in the constriction of the nanopore based on a second electrical response in the system, wherein the second electrical response depends on the identity of the second reporter element. The second reporter element may comprise one or multiple nucleotides in addition to other moieties, e.g., modifications attached to nucleotides. The second electrical response may be an ionic current through the nanopore. In some embodiments, the modification attached to the nucleotide also translocates through the nanopore. In some embodiments, the voltage is configured to be only sufficient to translocate one nucleotide through the nanopore at a time. In some embodiments, the second electrical response depends on the modification attached to nucleotides in the second reporter element. In some embodiments, the voltage is configured to be sufficient to translocate a plurality of nucleotides through the nanopore sequentially.

In some embodiments, the method may further comprise monitoring an electrical response in the system when applying the voltage to determine that only the first reporter element translocates through the nanopore, and ceasing the voltage after the first reporter element has translocated through the nanopore.

In some embodiments, provided herein is a method for determining a sequence of a target polynucleotide in a nanopore-based sequencing system, the method comprising: providing a target polynucleotide including nucleotides, wherein each nucleotide is attached to a modification, wherein the modification includes an arresting construct configured to arrest the target polynucleotide relative to a nanopore; and applying a driving voltage to translocate one or more portions of the target polynucleotide through a nanopore; measuring a current of the nanopore continuously during translocation; and identifying the sequence of the target polynucleotide by correlating the measured current to the identity of the nucleotides.

In another aspect, kits used for performing the disclosed methods are provided. The kit may comprise one or more cyclic loop nucleotides or nucleotides with any of the modifications described in the present disclosure.

In yet another aspect, systems or devices configured to determine a sequence of a target polynucleotide using the any of the disclosed methods are provided.

In some aspects, the techniques described herein relate to a method, wherein providing the target polynucleotide includes synthesizing a daughter strand based on a template polynucleotide using nucleotides with modifications, wherein the modifications are covalently attached to the nucleotides.

In some aspects, the techniques described herein relate to a method, wherein providing the target polynucleotide includes: synthesizing a daughter strand based on a template polynucleotide using nucleotides with modifications, wherein the modifications are covalently attached to the nucleotides; and cleaving the daughter strand to generate the target polynucleotide having an elongated polynucleotide strand.

In some aspects, the techniques described herein relate to a method, wherein the driving voltage is kept constant during the translocation.

In some aspects, the techniques described herein relate to a method, wherein the measured current is dependent on the reporter element or the nucleotide passing through the nanopore.

In some aspects, the techniques described herein relate to a method, wherein the arresting construct includes a polymer selected from the group consisting of a linear synthetic hydrophilic polymer, linear synthetic hydrophobic polymer, linear polynucleotide, linear polypeptide, branched polymer, dendritic polymer, cyclic polymer, fluoroalkyl, rigid conjugated chromophores, and rigid macrocycles.

In some aspects, the techniques described herein relate to a method, wherein the arresting construct includes a covalent coupling between the polymer and the corresponding nucleotide or the cyclic loop.

In some aspects, the techniques described herein relate to a method, wherein the covalent coupling is selected from the group consisting of amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene.

In some aspects, the techniques described herein relate to a method, wherein the linear synthetic hydrophilic polymer is selected from the group consisting of polyethyleneglycol, polyvinylalcohol, polyacrylamide, polyvinylpyrrolidone, polystyrenesulfonate, polyethyleneimine, and a combination thereof.

In some aspects, the techniques described herein relate to a method, wherein the linear synthetic hydrophobic polymer is selected from the group consisting of polylactic acid, polymethymethacrylate, polystyrene, and a combination thereof.

In some aspects, the techniques described herein relate to a method, wherein the linear polynucleotide is a homopolymer of a natural nucleotide, a homopolymer of an unnatural nucleotide, a mixed sequence polymer of natural nucleotides, or a mixed sequence polymer of unnatural nucleotides.

In some aspects, the techniques described herein relate to a method, wherein the linear polypeptide includes one or more types of amino acids.

In some aspects, the techniques described herein relate to a method, wherein the branched polymer includes two or more branches.

In some aspects, the techniques described herein relate to a method, wherein the cyclic polymer has 3 or more repeating units.

In some aspects, the techniques described herein relate to a method, each repeating unit is a small molecule, a nucleotide, or an amino acid.

In some aspects, the techniques described herein relate to a method, wherein the repeating units are the same.

In some aspects, the techniques described herein relate to a method, wherein at least two of the repeating units are different.

In some aspects, the techniques described herein relate to a system for determining a sequence polynucleotide or oligonucleotide according to methods disclosed herein.

In some aspects the techniques described herein relate to a cyclic loop nucleotide comprising a cyclic loop modification bridging a nucleobase and a phosphate group, wherein the cyclic loop modification comprises a reporter encoding the identity of the nucleobase, and an arresting construct adjacent to the reporter.

In some aspects the techniques described herein relate to a cyclic loop nucleotide wherein the arresting construct is adjacent to the reporter.

In some aspects the techniques described herein relate to a cyclic loop nucleotide having one of the following structures:

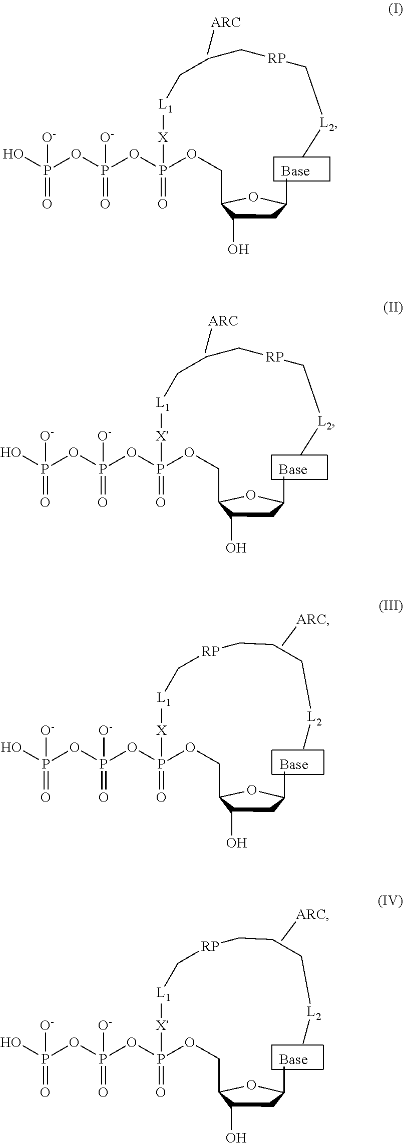

wherein: X is —O—, —CH₂—, —NSO₂—, —NH—,

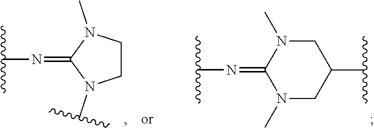

### X′ is —S—, ═N—SO₂—; ═NH—CO—, or

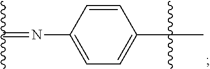 [SMILES: *c1ccc(N=*)cc1]

Base is the nucleobase; L₁ and L₂ are each a linking group; RP is the reporter encoding the nucleobase; and ARC is the arresting construct.

In some aspects the techniques described herein relate to a cyclic loop nucleotide wherein the ARC is covalently attached to the cyclic loop via a covalent coupling selected from amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene.

Additional details of exemplary nanopore sequencing devices which can be used with the disclosed technology, and methods of operating the devices, can be found in U.S. Provisional Patent Application Nos. 63/200,868 (WO 2022/005780) and 63/169041, the entirety of each of the disclosures is incorporated herein by reference.

It is to be understood that any features of the device and/or of the array disclosed herein may be combined together in any desirable manner and/or configuration. Further, it is to be understood that any features of the method of using the device may be combined together in any desirable manner. Moreover, it is to be understood that any combination of features of this method and/or of the device and/or of the array may be used together, and/or may be combined with any of the examples disclosed herein. Still further, it is to be understood that any feature or combination of features of any of the devices and/or of the arrays and/or of any of the methods may be combined together in any desirable manner, and/or may be combined with any of the examples disclosed herein.

It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 illustrates an example of controlled DNA translocation using modifications.

FIG. 2 illustrates an exemplary workflow for modification controlled nanopore sequencing.

FIG. 3 illustrates an exemplary library preparation process using modified nucleotides.

FIG. 4 illustrates an exemplary DNA insertion process to initiate nanopore sequencing.

FIG. 5 illustrates an exemplary DNA translocation process during modification controlled nanopore sequencing.

FIG. 6 illustrates an exemplary model system for screening arresting constructs.

FIG. 7 shows results from an experiment using the exemplary model system illustrated in FIG. 6.

FIG. 8 shows examples of linear, synthetic hydrophilic polymer arresting constructs.

FIG. 9 shows examples of polynucleotide arresting constructs.

FIG. 10 shows examples of branched arresting constructs.

FIG. 11 shows additional examples of branched arresting constructs.

FIG. 12 shows additional examples of branched arresting constructs.

FIG. 13 shows examples of cyclic arresting constructs.

FIG. 14 shows embodiments of arresting constructs with various properties.

FIG. 15 shows examples of common DNA base methylation sites of the exocyclic amine at the sixth position of adenine and the fifth carbon on the cytosine ring.

FIG. 16 illustrates an exemplary process of modifying a dsDNA using DNA methyltransferase (MTase) and SAM analogues.

FIG. 17 shows an exemplary structure of a SAM analogue having a modification.

FIG. 18 shows examples of arresting constructs which can be utilized in the exemplary structure shown in FIG. 17. Top: linear PEG. Bottom: branched PEG.

FIG. 19 shows an example of a SAM analogue having a PEG4 group conjugated via Cu-click reaction.

FIG. 20 illustrates an exemplary process of modifying a 5-methylcytosine using CMD1.

FIG. 21 illustrates an exemplary cyclic loop modification.

FIG. 22 illustrates an exemplary sequence comprising a plurality of cyclic loop modified nucleotides.

FIG. 23A-23C illustrates experimental assays and average dwell time of a cyclic loop modified construct at different driving voltage.

FIG. 24A-24C illustrates experimental results and current traces of a cyclic loop modified construct.

FIG. 25A-25D illustrates experimental results and current traces of a cyclic loop modified construct.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

### Definitions

All technical and scientific terms used herein have the same meaning as commonly understood to one of ordinary skill in the art to which this disclosure belongs unless clearly indicated otherwise.

As used herein, the singular forms “a”, “and”, and “the” include plural referents unless the context clearly dictates otherwise. Thus, for example, reference to “a sequence” may include a plurality of such sequences, and so forth.

The terms comprising, including, containing and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

As used herein, the term “nanopore” is intended to mean a hollow structure discrete from, or defined in, and extending across the membrane. The nanopore permits ions, electric current, and/or fluids to cross from one side of the membrane to the other side of the membrane. For example, a membrane that inhibits the passage of ions or water-soluble molecules can include a nanopore structure that extends across the membrane to permit the passage (through a nanoscale opening extending through the nanopore structure) of the ions or water-soluble molecules from one side of the membrane to the other side of the membrane.

The diameter of the nanoscale opening extending through the nanopore structure can vary along its length (i.e., from one side of the membrane to the other side of the membrane), but at any point is on the nanoscale (i.e., from about 1 nm to about 100 nm, or to less than 1000 nm). Examples of the nanopore include, for example, biological nanopores, solid-state nanopores, and biological and solid-state hybrid nanopores. In some embodiments, a nanopore refers to a pore having an opening with a diameter at its most narrow point of about 0.3 nm to about 2 nm. For example, a nanopore may be a solid-state nanopore, a graphene nanopore, an elastomer nanopore, or may be a naturally-occurring or recombinant protein that forms a tunnel upon insertion into a bilayer, thin film, membrane, or solid-state aperture, also referred to as a protein pore or protein nanopore herein (e.g., a transmembrane pore). If the protein inserts into the membrane, then the protein is a tunnel-forming protein.

As used herein, the term “diameter” is intended to mean a longest straight line inscribable in a cross-section of a nanoscale opening through a centroid of the cross-section of the nanoscale opening. It is to be understood that the nanoscale opening may or may not have a circular or substantially circular cross-section (the cross-section of the nanoscale opening being substantially parallel with the cis/trans electrodes). Further, the cross-section may be regularly or irregularly shaped.

As used herein, the term “biological nanopore” is intended to mean a nanopore whose structure portion is made from materials of biological origin. Biological origin refers to a material derived from or isolated from a biological environment such as an organism or cell, or a synthetically manufactured version of a biologically available structure. Biological nanopores include, for example, polypeptide nanopores and polynucleotide nanopores.

As used herein, the term “polypeptide nanopore” is intended to mean a protein/polypeptide that extends across the membrane, and permits ions, electric current, polymers such as DNA or peptides, or other molecules of appropriate dimension and charge, and/or fluids to flow therethrough from one side of the membrane to the other side of the membrane. A polypeptide nanopore can be a monomer, a homopolymer, or a heteropolymer. Structures of polypeptide nanopores include, for example, an a-helix bundle nanopore and a B-barrel nanopore. Example polypeptide nanopores include a-hemolysin, Mycobacterium smegmatis porin A (MspA), gramicidin A, maltoporin, OmpF, OmpC, PhoE, Tsx, F-pilus, etc. The protein a-hemolysin is found naturally in cell membranes, where it acts as a pore for ions or molecules to be transported in and out of cells. Mycobacterium smegmatis porin A (MspA) is a membrane porin produced by Mycobacteria, which allows hydrophilic molecules to enter the bacterium. MspA forms a tightly interconnected octamer and transmembrane beta-barrel that resembles a goblet and contains a central pore.

A polypeptide nanopore can be synthetic. A synthetic polypeptide nanopore includes a protein-like amino acid sequence that does not occur in nature. The protein-like amino acid sequence may include some of the amino acids that are known to exist but do not form the basis of proteins (i.e., non-proteinogenic amino acids). The protein-like amino acid sequence may be artificially synthesized rather than expressed in an organism and then purified/isolated.

The nanopores disclosed herein may be hybrid nanopores. A “hybrid nanopore” refers to a nanopore including materials of both biological and non-biological origins. For example, a hybrid nanopore may include biological materials such as polypeptides and/or polynucleotides adjacent and/or conjugated to non-biological materials such as semiconductors and/or other solid-state aspects. Thus a hybrid nanopore may be a polypeptide-solid-state hybrid nanopore and a polynucleotide-solid-state nanopore.

The application of the potential difference across a nanopore may force the translocation of a nucleic acid through the nanopore. One or more signals are generated that correspond to the translocation of the nucleotide through the nanopore. Accordingly, as a target polynucleotide, or as an oligonucleotide or mononucleotide or a probe derived from the target polynucleotide or mononucleotide, transits through the nanopore, the current across the membrane changes due to base-dependent (or probe dependent) blockage of the constriction, for example. The signal from that change in current can be measured using any of a variety of methods. Each signal is unique to the species of nucleotide(s) (or probe) in the nanopore, such that the resultant signal can be used to determine a characteristic of the polynucleotide or oligonucleotide. For example, the identity of one or more species of nucleotide(s) (or probe) that produces a characteristic signal can be determined.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides are natural nucleotides, but it is to be further understood that non-natural nucleotides, modified nucleotides or analogs of the aforementioned nucleotides can also be used.

As used herein, the term “signal” is intended to mean an indicator that represents information. Signals include, for example, an electrical signal and an optical signal. The term “electrical signal” refers to an indicator of an electrical quality that represents information. The indicator can be, for example, current, voltage, tunneling, resistance, potential, voltage, conductance, or a transverse electrical effect. An “electronic current” or “electric current” refers to a flow of electric charge. In an example, an electrical signal may be an electric current passing through a nanopore, and the electric current may flow when an electric potential difference is applied across the nanopore.

As used herein, the term “driving force” or “driving voltage” is intended to mean an electrical current that allows at least a portion of the polynucleotide or oligonucleotide to translocate through the nanopore. In some embodiments, the electric current may flow when an electric potential difference is applied across the nanopore.

As used herein, the term “holding force” is intended to mean a resistance that slows and/or stops a polynucleotide or oligonucleotide to translocate through the nanopore. In some embodiments, the holding force is overcome by the application of a driving force. Thus, the driving force overcomes/overrides the resistance that slows and/or stops a polynucleotide, thereby allowing the polynucleotide to translocate through the nanopore.

As used herein, the term “modification” is intended to mean a moiety attached to a nucleotide. A modification may comprise an arresting construct or a cyclic loop moiety that further comprises an arresting construct. A modification can be attached to any part of the nucleotide and can also be attached to the nucleotide at two locations forming a loop.

As used herein, the term “arresting construct” may provide a resistance (in the form of a “holding force”) that slows and/or stops a polynucleotide, oligonucleotide, or mononucleotide to translocate through the nanopore unless the resistance due to the arresting construct is overcome by a “driving force.” The resistance provided by the arresting construct is due to a property of the arresting construct (e.g., size, geometry, and/or non-covalent interaction with the nanopore). An arresting construct can operate as a ratchet or a brake for the polypeptide translocation through a nanopore.

As used herein, the term “arrest” is intended to mean stop and/or slow down. For example, when the translocation of a polynucleotide through a nanopore is arrested, the relative motion of the polynucleotide with respect to the nanopore may come to rest or may continue but with a slower speed. An arresting construct that arrests nucleotide translocation through a nanopore may serve to stop or slow down translocation compared to an unmodified nucleotide.

As used herein, a “reporter element” includes what are known as “tags” or “labels.” A reporter element may comprise one or more nucleotides or polymers. Four reporter elements may encode each of the nucleobases, which produce distinguishable signals (fingerprint/signature) when passing through the readhead of a nanopore. Multiple reporters or reporter signals may also encode a nucleobase. A reporter may be comprised of two or more sub-reporters.

The term “nucleic acid” or “polynucleotide” refers to a deoxyribonucleotide or ribonucleotide polymer in either single-or double-stranded form, and unless otherwise limited, encompasses known analogs of natural nucleotides that hybridize to nucleic acids in manner similar to naturally occurring nucleotides, such as peptide nucleic acids (PNAs) and phosphorothiolate DNA. Unless otherwise indicated, a particular nucleic acid sequence includes the complementary sequence thereof. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP. Polynucleotide and oligonucleotide may be interchangeable herein.

As used herein, “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, LO), all herein incorporated by reference in their entireties.

As used herein, a “peptide” refers to two or more amino acids joined together by an amide bond (that is, a “peptide bond”). Peptides comprise up to or include 50 amino acids. Peptides may be linear or cyclic. Peptides may be α, β, γ, δ, or higher, or mixed. Peptides may comprise any mixture of amino acids as defined herein, such as comprising any combination of D, L, α, β, γ, δ, or higher amino acids.

As used herein, a “moiety” is one of two or more parts into which something may be divided, such as, for example, the various parts of a tether, a molecule or a probe.

As used herein, “cis” refers to the side of a nanopore opening through which an analyte or modified analyte enters the opening. The “cis” side may be dependent upon the applied positive or negative voltage.

As used herein, “trans” refers to the side of a nanopore opening through which an analyte or modified analyte (or fragments thereof) exits the opening.

The aspects and examples set forth herein and recited in the claims can be understood in view of the above definitions.

### Introduction

The disclosed technology relates to systems and methods for strand-based nucleic acid nanopore sequencing approaches, e.g., polynucleotide or DNA strand-based nanopore sequencing. A sequence of the strand may be determined by reading the base-specific changes in the ionic current through the nanopore as a strand of polynucleotide or oligonucleotide passes through a constriction of the nanopore. However, the sequencing readouts depend not only on the size of the constriction in the nanopore constriction but also on the rate of translocation of polynucleotide through the nanopore (i.e., translocation speed).

Translocation of a polynucleotide may be driven by a voltage bias which drive the movement of bases through a nanopore readhead. In some embodiments, the voltage bias is constant, which is within a set range of predefined voltages. In some embodiments, the voltage bias cycles up to a reading or driving voltage, wherein the voltage is sufficient to drive translocation. Rates of translocation under effective driving voltages in nanopores may be in the range of 10⁶ bases/sec. In this range, the translocation rates may be too fast and can adversely affect the reading accuracy. Due to intrinsic limitations of the current detection electronics, it is desirable to reduce translocation speeds by several orders of magnitude to achieve single base resolution. Translocation may be driven by a constant voltage bias (e.g. automatic advancement), or by transient voltage changes (e.g. pulses) which drive the movement of bases through a nanopore readhead.

Some methods of reducing translocation speed rely on so-called motor enzymes (e.g., helicases and polymerases). However, these methods have several drawbacks. First, the translocation rate is under the control of the enzyme. Each translocation event may be occurring too rapidly to yield a suitable signal-to-noise ratio and resulting in sequencing errors. Each translocation event may not occur at a precise, regular timing. Enzymatic motors may result in irregular translocation even when engineered to have a more regular motion. Second, enzymes have other drawbacks including back-stepping and energy requirements that lead to errors and complicate the system.

In some instances, transient voltage pulses may advance translocation of the polynucleotide or oligonucleotide through a nanopore readhead. In certain embodiments, an arresting construct may halt translocation through the nanopore when sequencing a polynucleotide or oligonucleotide. However, under certain circumstances, such as under a pulsed voltage regime, translocation of the polynucleotide through the nanopore may result in a skip or stall. For example, a skip may result in a signal from a reporter not being detected and a stall may result in the same reporter being detected after a pulse which was intended to further translocate the polynucleotide. In some cases, when a polynucleotide translocation results in a skip, a voltage pulse may translocate multiple bases and/or arresting constructs, thereby generating a transient, or even non-existent signal as the multiple bases pass through the readhead. The effect of skipping then, truncates and produces errors in measured sequences of a target polynucleotide. In some embodiments, controlled movement of certain modified nucleobases may skip at a rate of 1-10%. Alternatively, when stalling, multiple transient voltage pulses may be required to advance a base in the readhead of a nanopore, thereby slowing overall advancement of the polynucleotide.

Under a pulsed voltage regime, an average of 3-10 voltage pulses could be applied before successful advancement of a base across the nanopore readhead. Either skipping or stalling may therefore increase error rates when sequencing a nucleic acid. Some methods explore the use of DNA polymerase-nanopore conjugate for sequencing a template nucleic acid, and the maximal achievable read length and sequencing accuracy are affected by the stability of the polymerase-template complex. In some instances, a constant voltage bias advancement may be utilized to sequence a nucleic acid, wherein controlled movement of modified nucleobases occur without the need for voltage pulses, or wherein the background voltage is sufficient to drive bases through the nanopore. In some embodiments where a constant voltage bias is utilized, a system of the preset disclosure may detect changes in reporter signal regardless of duration.

In some embodiments, the present disclosure is related to a strategy that addresses the second consideration noted above, i.e., translocation speed. A target polynucleotide or oligonucleotide (such as a modified DNA) may be provided with modifications designed to slow down the translocation speed. A modification may comprise an arresting construct. In some embodiments, a modification may include a cyclic loop moiety comprising an arresting construct. In some embodiments, provided herein are a selection of arresting constructs and understanding of their interactions with nanopores. In some embodiments, provided herein are pore mutation or modification to provide enhanced arresting construct-based control of nucleic acid translocation. In some embodiments, provided herein are synthesis of novel modifications with fine-tuned properties for enhanced nucleic acid translocation behavior. In some embodiments, provided herein are chemistry for selective conjugation of modifications.

In some embodiments, in order to enable controlled single-base translocation of polynucleotide or oligonucleotide (such as DNA) through a nanopore and to enhance the reading accuracy and efficiency of the polynucleotide sequence, provided herein is the concept of attaching “modifications” on the nucleotides of the daughter strand for insertion through a nanopore. In some embodiments, modifications permit controlled movement of polynucleotide through the nanopore. The modification may comprise an arresting construct. In some embodiments, the modification may comprise a cyclic loop moiety, and the cyclic loop moiety further comprises an arresting construct. Without being limited by any particular theory, the size, geometry, and charges on arresting constructs have direct impact on the speed of polynucleotide translocation. In some embodiments, owing to their sizes and interactions with the nanopore, the arresting construct slows and/or stops polynucleotide translocation under electrical conditions that are suitable for reading the polynucleotide. In some embodiments, application of voltage results in a driving force and/or a change in the electrical conditions that are suitable for translocation of the polynucleotide through the nanopore. The driving force overcomes a holding force that holds the polynucleotide in place as a result of interactions between the arresting construct and the nanopore.

This method of controlled translocation may have several significant advantages over traditional strand-based approaches. In some embodiments, stopping and/or slowing target polynucleotide or oligonucleotide translocation by modifications greatly enhances temporal resolution of bases resulting in more accurate base identification. In some embodiments, control of translocation mitigates the homopolymer problem (that accurately discerning a sequence of repeating base is difficult) typically associated with strand-based nanopore sequencing methods. In some embodiments, base-calling algorithms could be simplified because the sufficiently regular timing between base signals may permit algorithms to focus on relevant portions of the signal stream.

In some embodiments, controlled polynucleotide translocation is obtained by attaching nucleotides with modification moieties, including arresting constructs, which owing to their physical-chemical properties, slow or arrest the translocation when encountering the nanopore. In some embodiments, by using cleavable sites along the polynucleotide backbone while conjoining adjacent nucleobases with a barcoding region, the disclosed technology allows the distance between adjacent nucleobases to be increased and negates the need to deconvolute a large number of signals. In some embodiments, the disclosed technology allows one elongated nucleotide to reside in the readhead at any point in time, successfully reducing the diversity of reads to 4 (A, T, C and G), enabling more accurate sequencing at a lower cost. In some embodiments, the disclosed technology provides high throughput, cheaper and more accurate polynucleotide sequencing. In some embodiments, no change in voltage bias is required to advance a nucleotide through the readhead of a nanopore. In some embodiments, a change in voltage bias is required to advance a nucleotide through the readhead of a nanopore. Therefore, in some embodiments, a kit for polynucleotide sequencing containing at least nucleotides with modifications as disclosed herein is provided.

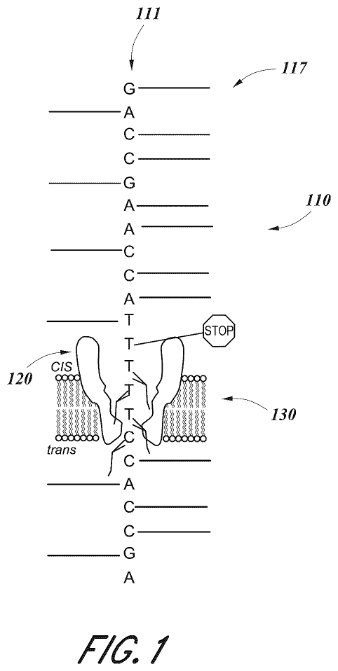

In some embodiments, modifications can be used to control polynucleotide translocation by modifying every nucleotide on a strand to carry a modification, where the modifications will be moving together with the nucleotides through the nanopore. As shown in FIG. 1, a protein nanopore 120 is deposited in a lipid bilayer 130. A single-stranded DNA 110 is passing, from the “cis” side, through the nanopore 120, to the “trans” side. The DNA 110 comprises nucleotides having modifications attached to them. For example, the nucleotide 111 (the “G” base) is attached to a modification 117, wherein the modification comprises an arresting construct. Without being bound by theory, when an arresting construct on a nucleotide comes into contact with a portion of the nanopore 120, it can “stop” or “slow” the DNA translocation. In the particular example shown in FIG. 1, every nucleotide of the DNA 110 is attached to a modification. However, in some examples, the nucleotides in the head and/or tail portion of a DNA may not be decorated with modifications. In some examples, not every nucleotide is decorated, e.g., only one in every two, three, four or five nucleotides is decorated with a modification comprising an arresting construct.

In some embodiments, the present disclosure is related to arresting constructs to stop and/or slow down DNA translocation. In some embodiments, the present disclosure is related to ways in which arresting construct properties can be adjusted to enhance and/or fine-tune their arresting ability. In some embodiments, the present disclosure is related to a system for determining a sequence of a polynucleotide using the method disclosed herein. In some embodiments, no change in voltage bias is required to advance a nucleotide through the readhead of a nanopore. In some embodiments, a change in voltage bias is implemented to advance a nucleotide through the readhead of a nanopore.

### Operation

The polynucleotide sequencing techniques described herein utilize a nanopore, which can provide a path for an ionic electrical current. For example, as the polynucleotide traverses through the nanopore, it influences the electrical current through the nanopore. In some embodiments, each passing nucleotide, or series of nucleotides, that passes through the nanopore can yield a characteristic electrical current. In some embodiments, each recorder elements that pass through the nanopore can also yield a characteristic electrical current. These characteristic electrical currents of the translocating polynucleotide can be recorded to determine the sequence of the polynucleotide.

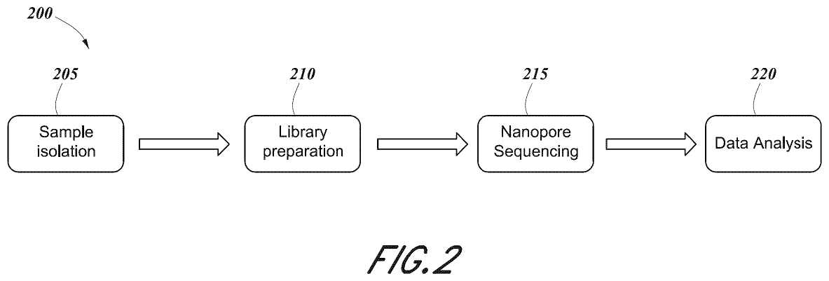

FIG. 2 illustrates an exemplary workflow 200 for the nanopore sequencing method described herein. The workflow 200 starts at step 205, isolation of sample polynucleotide (such as DNA) from biological sources using suitable extraction methods. After isolation of sample polynucleotide, the workflow moves to step 210, where the sample polynucleotide is subjected to a library preparation process which comprises using the sample polynucleotide as templates for synthesizing new daughter strands using modified polynucleotides with modifications. During this step, the modifications are introduced on the newly synthesized polynucleotide strand. In some embodiments, the modification comprises a cyclic loop structure with an arresting construct. The nucleotides may include sites where one or more chemical bonds may be broken to form elongated polynucleotide.

After library preparation, the workflow moves to the nanopore sequencing step 215, where the modification-modified polynucleotide in the library is translocated through the nanopore, and data during translocation is collected to be used for determining the base identities. After the nanopore sequencing step, the workflow moves to the data analysis step 220, where base calls are made based on the data collected. In some embodiments, the workflow 200 of FIG. 2 is a part of a sequencing cycle.

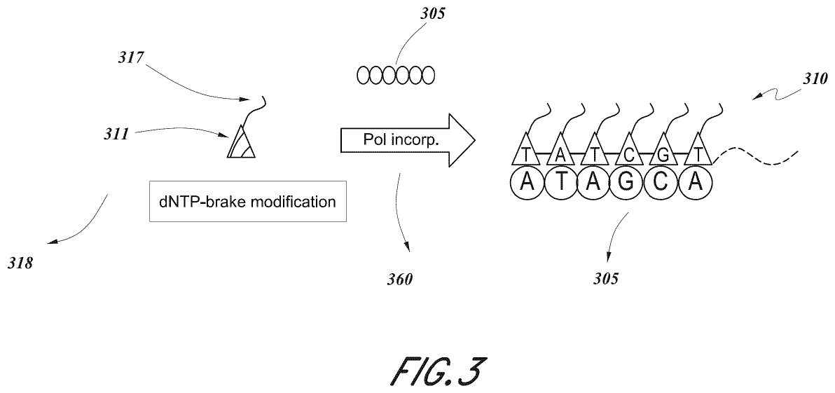

In some embodiments, producing a polynucleotide with modifications is performed by incorporating modification-tagged nucleotides (i.e., modified nucleotides) to a daughter strand being synthesized by a polymerase, for example, as illustrated in FIG. 3. As shown in the exemplary library preparation process using modified nucleotides of FIG. 3, polymerase 360 is synthesizing from a template polynucleotide 305 of the isolated sample polynucleotide. During library generation, sample DNA is converted into a library of “daughter strands” 310 via polymerase incorporation of modification-attached nucleotides 318, or using a ligase, for example. Each modification-attached nucleotide 318 includes a nucleotide 311 and a modification 317. In some iterations of nanopore sequencing, the daughter strand 310 can also include a unique barcode (e.g., reporter element) on each base to improve single base resolution and sequencing accuracy. In some iterations, a leader oligo is appended to the daughter strand 310 to provide direction specific insertion in the nanopore.

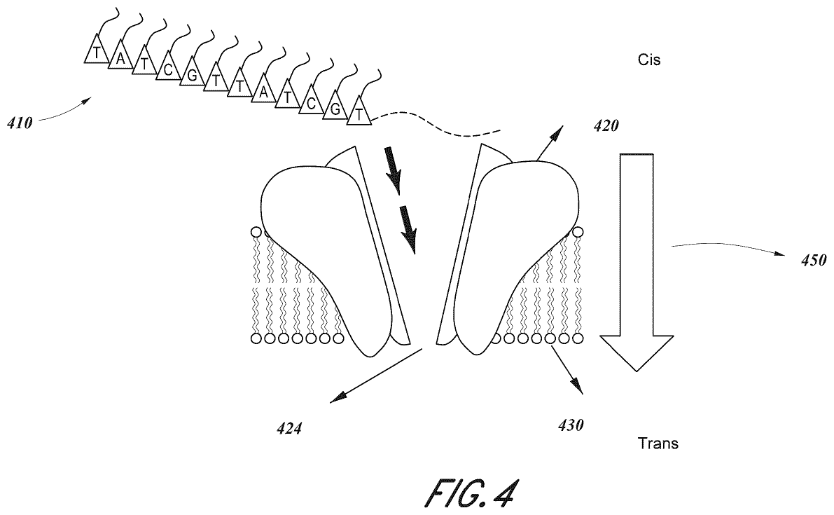

FIG. 4 illustrates that a voltage 450 is applied to introduce a target DNA 410 into a nanopore 420. In the particular example of FIG. 4, the protein pore 420 having a constriction region 424 is inserted in a lipid bilayer 430. In other examples, a solid-state pore is directly fabricated in a synthetic membrane, which can be modified for DNA translocation and can be used for nanopore sequencing. The nanopore 420 separates two chambers denoted as cis and trans. Both chambers are filled with a suitable electrolyte solution and a voltage 450 may be applied across the nanopore. The target polynucleotide 410, e.g. a daughter strand from the library generation step, can be added to the cis, trans or both chambers of the nanopore. Introduction of polynucleotide into the nanopore 420 can be achieved by application of a capture voltage 450. In some embodiments, the nanopore may be deposited such that the constriction region of the nanopore may be closer to the trans chamber. In other embodiments, the nanopore may be deposited such that the constriction region of the nanopore may be closer to the cis chamber.

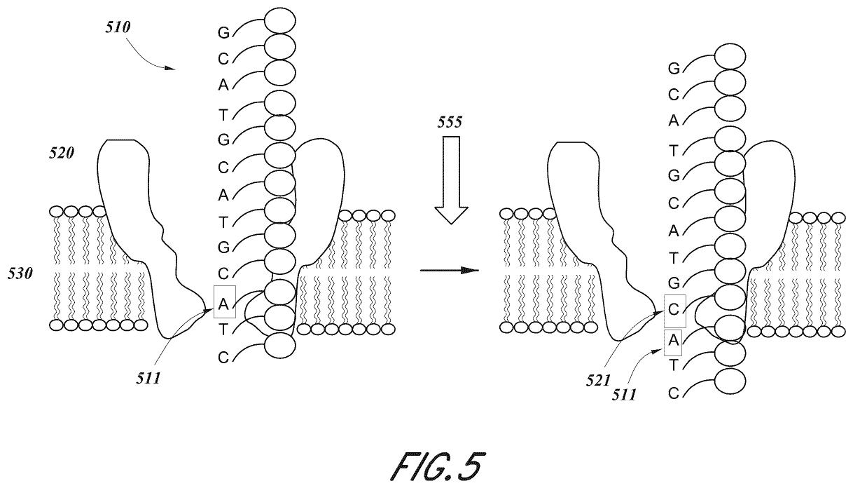

FIG. 5 illustrates a method of detection of nucleotides and controlled translocation of the polynucleotide through a nanopore by a suitable driving voltage 555. The left-hand side of FIG. 5 shows that a protein nanopore 520 is deposited in a lipid bilayer 530. As the daughter strand polynucleotide 510 translocates through the nanopore 520, one or more arresting constructs may encounter and interact with the nanopore 520, arresting the translocation of polynucleotide 510.

To identify the nucleotide 511 (base “A”) positioned in the constriction of the nanopore 520, in some embodiments, a characteristic ionic blockage current that depends on the nucleotide 511 or the arresting construct attached to the nucleotide can be recorded by the system. In other examples, the characteristic ionic blockage current may depend on two, three, four or five nucleotides positioned near the constriction of the nanopore. The characteristic ionic blockage current may further depend on the arresting constructs attached to the two, three, four or five nucleotides positioned near the constriction. In some embodiments, the characteristic ionic blockage current may depend on the modification attached to the nucleotide 511, such as the reporter element that is part of the modification.

After detection of the nucleotide 511, the next arresting constructs encounter the nanopore and the polynucleotide translocation arrests again. The right-hand side of FIG. 5 shows that the nucleotide 521 subsequent to the nucleotide 511 now sits in the constriction of the pore. The recorded characteristic ionic blockage current will now depend on the nucleotide 521. The characteristic ionic blockage current may further depend on the modification attached to the nucleotide 521.

### Modifications

Nanopores with constriction diameters in the subnanometer range are desired to prevent multiple strands or secondary structures from translocating through the pore. Another important dimension is the length of the constriction which determines the number of bases that contribute to the blockage current. Protein pores such as MspA with diameters as small as 1.2 nm have been used for distinguishing homopolymeric DNA. At its narrowest, the MspA constriction is 0.6 nm long, however the blockage current is determined by at least 4 nucleobases residing in the constriction. This means minimally 4\hat{ }4 (256) signals can arise based on the sequence of the 4-mer in the constriction.

In some embodiments, modifications may include an arresting construct. In some embodiments, modifications may include constituent elements to elongate or stretch adjacent nucleobases in a polynucleotide, such as cyclic loop group. Certain sites on a modification, or otherwise on a modified nucleotide, may have one or more chemical bonds broken to generate an elongated strand, which may serve to separate out signal generation when the elongated strand translocates through a nanopore readhead. In some embodiments, the cyclic loop may further include an arresting construct. In some embodiments, the cyclic loop may further include a reporter element or a reporter element comprising two or more sub-reporter elements. In some embodiments, the cyclic loop may also include a spacer, which can be used to adjust the spacing between the adjacent nucleotides, between the arresting construct and reporter element, or between the arresting construct of one nucleotide and the arresting construct of another nucleotide in the target polynucleotide.

The dwell time of an arresting construct in the constriction is expected to correlate with its physical and molecular size. Thus, attaching arresting constructs of various sizes and molecular weight to the nucleotides or the cyclic loop modification of a polynucleotide strand can affect the rate of polynucleotide translocation through the nanopore. Provided herein are various moieties that can be used as arresting constructs. The arresting construct are tunable to affect the translocation speed (e.g., to slow or arrest translocation) of polynucleotide through a nanopore. This can be done by varying the size and/or geometry of the modification moieties, and/or by changing the nature of interactions between the modification moieties and the nanopore.

### Arresting Constructs of Varying Sizes and Geometry

The size of the arresting construct can be modulated by increasing: i) physical length of the modification moiety, and ii) molecular weight of the modification moiety. In some embodiments, the arresting construct may comprise a linear polymer. Many of these polymers are commercially available, and with a wide variety of structures and chemical properties to choose from. The size and weight of the linear polymer can be tuned by changing the number of repeating units in the polymer chain (thus changing the molecular weight). In some embodiments, linear polymers may include, but not limited to, linear hydrophilic synthetic polymers, linear hydrophobic synthetic polymers, polynucleotides, peptides, and polypeptides.

In some embodiments, hydrophilic synthetic polymers may include, but not limit to, polyethyleneglycol, polyvinylalcohol, polyacrylamide, polyvinylpyrrolidone, polystyrenesulfonate, polyethyleneimine, and combinations thereof. Thus, an arresting construct comprising a hydrophilic synthetic polymer may be represented by the following structures:

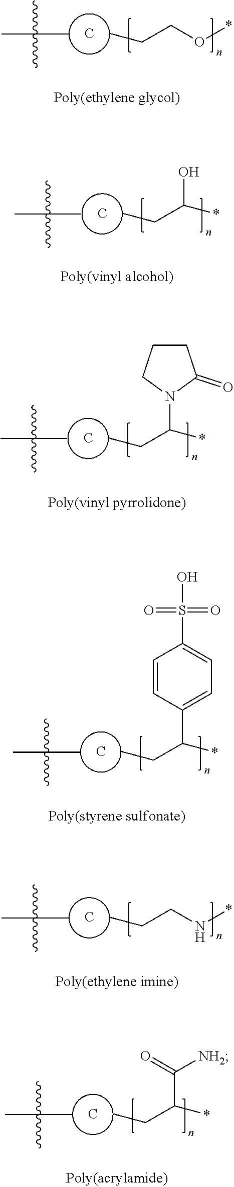

where n refers to the number of repeat units in the polymer chain,

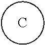

refers to a covalent coupling between the polymer and the nucleotide on the daughter strand, and * is an end group on the polymer chain. The end group on the polymer chain is not particularly limited and may be any proper polymer end group. For example, * may include, but not limit to, hydrogen, alkyl (e.g., methyl), halogen (e.g., bromide), thiol, amine, dithiobenzoate, dodecyltrithiocarbonate, phenylcarbamodithioate, or dimethylacetic acid. In some embodiments the polymer may be a continuous chain within the cyclic loop, as opposed to a branch off the cyclic loop. Thus, in some embodiments * may be a covalent coupling to another portion of the cyclic loop. In some embodiments, n is about 10 to about 200, about 10 to about 100, or about 100 to about 200. In some embodiments,

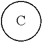

may comprise a coupling moiety selected from the group consisting of amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene, In some embodiments,

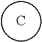

may further comprise a linker between the coupling moiety and the polymer. Some examples of arresting constructs comprising hydrophilic synthetic polymer are illustrated in FIG. 8. In some embodiments, the arresting construct may comprise a copolymer of 2 or more hydrophilic synthetic polymers.

In some embodiments, hydrophobic synthetic polymers may include polylactic acid, polymethymethacrylate, polystyrene, and a combination thereof. Thus, an arresting construct comprising a hydrophobic synthetic polymer may be represented by the following structures:

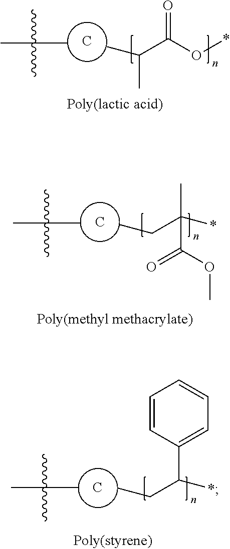

wherein n refers to the number of repeat units in the polymer chain,

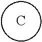

refers to a covalent coupling between the polymer and the nucleotide on the daughter strand, and * is an end group on the polymer chain. The end group on the polymer chain is not particularly limited and may be any proper polymer end group. For example, * may include, but not limit to, hydrogen, alkyl (e.g., methyl), halogen (e.g., bromide), thiol, amine, dithiobenzoate, dodecyltrithiocarbonate, phenylcarbamodithioate, dimethylacetic acid. In some embodiments, n is about 10 to about 200, about 10 to about 100, or about 100 to about 200. In some embodiments,

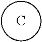

may comprise a coupling moiety selected from the group consisting of amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene. In some embodiments,

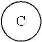

may further comprise a linker between the coupling moiety and the polymer. In some embodiments, the arresting construct may comprise a copolymer of 2 or more hydrophobic synthetic polymers, e.g., polylactic acid-co-polymethyl methacrylate, polystyrene-co-polymethyl acrylate, polystyrene-co-polylactic acid.

In some embodiments, arresting constructs can include DNA/RNA polynucleotides and other phosphate containing polymers. In some embodiments, polynucleotides may include homopolymers, mixed sequence polymers, 4-mer repeat sequences, poly a basic nucleotides, etc. In some embodiments, phosphate-containing polymers may include synthetic organic monomers connected by phosphate linkages. Complex, mixed base polynucleotides of varying length can be synthesized on automated synthesizers. Thus, an arresting construct comprising a polynucleotide or phosphate-containing polymers may be represented by the following structures:

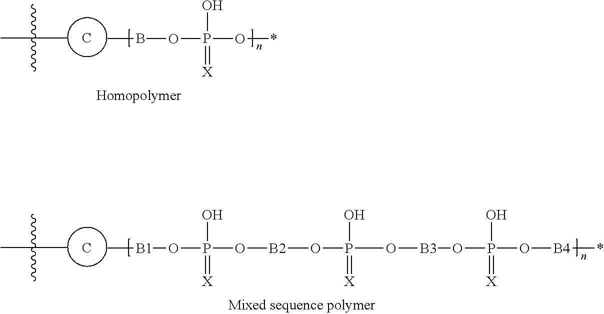

wherein n refers to the number of repeating units, B refers to natural or unnatural nucleotides and organic molecule repeating units; B1, B2, B3, B4 refer to a mixed sequence of natural or unnatural nucleotides and organic molecule repeating units; and X refers to heteroatom on the phosphate linkage where X═O or S,

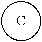

refers to a covalent coupling between the peptide/polypeptide and the nucleotide on the daughter strand, and * is an end group on the polymer chain. The end group on the polymer chain is not particularly limited, and may be any proper polymer end group. For example, * may include, but not limit to, hydroxyl, amine, phosphate and phosphorothioate. In some embodiments, n is about 1 to about 50, about 10 to about 40, or about 10 to about 30. In some embodiments,

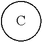

may comprise a coupling moiety selected from the group consisting of amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene. In some embodiments,

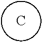

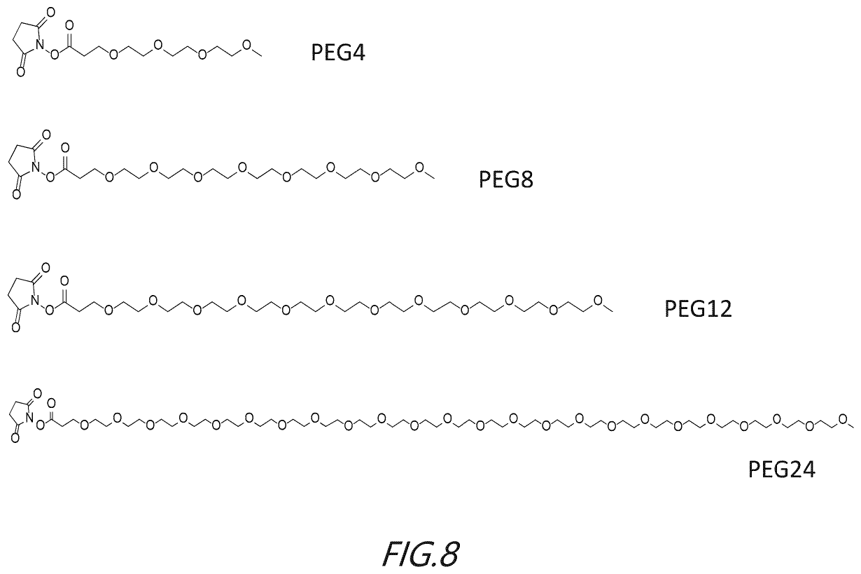

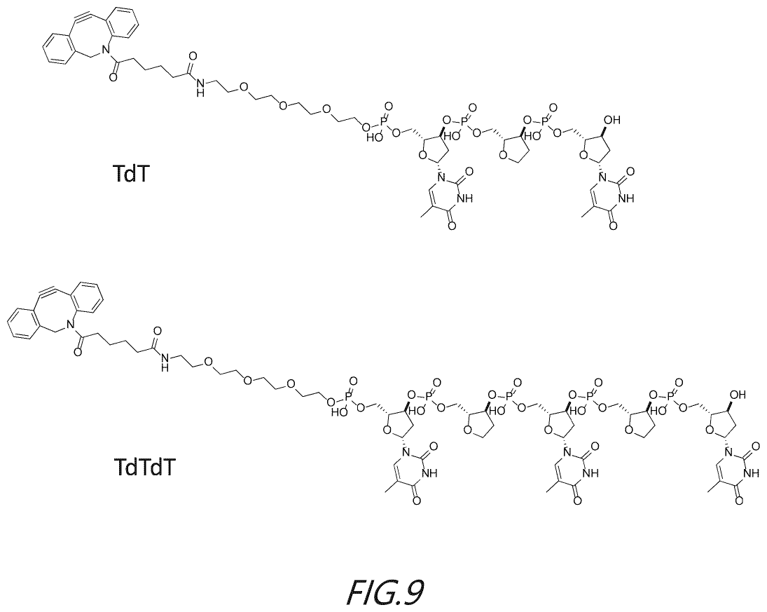

may further comprise a linker between the coupling moiety and the polymer. Some examples of arresting constructs comprising polynucleotide are shown in FIG. 9.

In some embodiments, peptides/polypeptides may include amino acids, a sequence of amino acids or polypeptides. Size of peptides/polypeptides can be modulated by i) changing the number of amino acids in the peptide sequence, ii) a judicial selection of individual amino acids with various side chains, or iii) chemical modification of amino acid residues (e.g., phosphorylation, sulfonation, conjugation of small molecules to the side chains). In some embodiments, non-limiting examples of small molecules may include polyvinyl alcohol, polyvinylpyrrolidone, polystyrene, polystyrene sulfonate, polymethylmethacrylate, polylactic acid, D-glucose, polyethyleneimine, polyacrylamide, glycouril, dialkoxybenezenes, polyethyleneglycol, polycarbonates, polymethymethacrylate or polyamides. Polypeptide arresting constructs can include one type of amino acid or a mixture of different types/varying sizes of amino acids. Thus, an arresting construct comprising a peptide/polypeptide may be represented by the following structures:

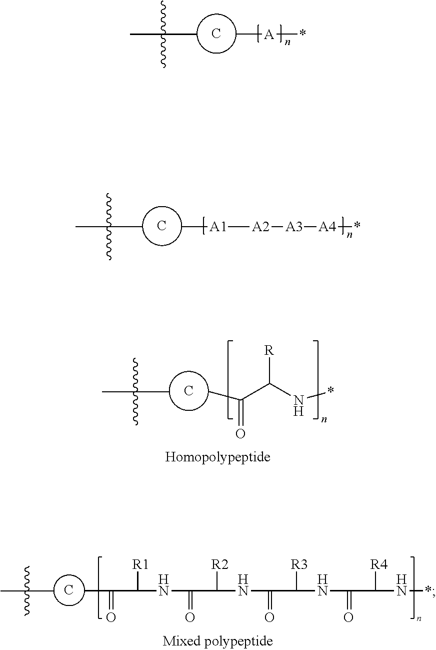

wherein n refers to the number of repeating units; A refers to the amino acid repeating unit in a homopolypeptide based arresting construct; R refers to the side chain residue of amino acid A; A1, A2, A3, A4 refer to the amino acids in a mixed polypetide arresting construct; R1, R2, R3, R4 refer to the side chain residues of A1, A2, A3, A4, respectively,

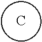

refers to a covalent coupling between the peptide/polypeptide and the nucleotide on the daughter strand, * is an end group on the peptide/polypeptide, and * is an end group on the peptide/polypeptide. The end group on the peptide/polypeptide chain is not particularly limited and may be any proper peptide/polypeptide end group. In some embodiments, * is amide, acetyl, carboxylic acid or amine. In some embodiments, n is about 1 to about 50, about 10 to about 40, or about 10 to about 30. In some embodiments,

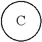

may comprise a coupling moiety selected from the group consisting of amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene. In some embodiments,

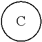

may further comprise a linker between the coupling moiety and the peptide/polypeptide. In some embodiments, more than 4 types of amino acids may be present in a mixed polypeptide subunit. In other embodiments, 2 or 3 types of amino acids may be present in a mixed polypeptide subunit.

In some embodiments, two or more different types of polymers may be linked together to form an arresting construct. As such, the arresting construct may comprise a copolymer of various polymer types. In some embodiments, the arresting construct may comprise hydrophilic synthetic polymer(s), hydrophobic synthetic polymer(s), polynucleotide(s), and/or peptide/polypeptide(s) that are connected in series. A linker may be present between any two connected polymers. A linker may also be a part of covalent coupling

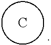

While the size of linear arresting constructs can be easily tuned by connecting multiple constructs in series (including different types of modifications), flexibility of such constructs allows the adoption of multiple conformations in solution. Some conformations of linear constructs promote or enhance translocation ability through the pore thereby reducing their residence time in the constriction. In some scenarios, large linear arresting constructs may bypass the pore completely without slowing or arresting the translocation of DNA. Thus, in addition to size, it may be helpful to tune the geometry of the arresting construct to improve its stopping capability. The geometry of an arresting construct can be modulated by i) increasing cross-sectional diameter of the arresting construct moieties or by ii) increasing structural rigidity of the modification moieties.

In some embodiments, cross sectional diameter of the arresting construct moieties can be modified by introducing branching elements in the molecular design or by using a cyclic moiety. In some embodiments, the cross-sectional diameter of arresting construct can range from about 1.5 nm, 2.0 nm, 2.5 nm, 3.0 nm to about 3.5 nm. In some embodiments, the linear arresting construct can be modified to introduce one or more branched structures. In some embodiments, the cross sectional diameter of an arresting construct moiety can be modulated by varying the number of branches/polymer arms from the branching point in the structure of the arresting construct. In some embodiments, arresting constructs can include multiple branching points to mimic a dendritic structure. In some embodiments, arresting constructs can include 1, 2, 3, 4, 5, 6, 7, 8, 9, 10, or more branching points to mimic dendritic structures.

In some embodiments, the arresting construct comprises a branched polymer. The branched polymer may have 2, 3, or 4 branches or polymer arms. In some embodiments, the branched polymer may have 1 or more branching points. For example, each of the branches on the first branching point (i.e., first generation branching point) may further includes another branching point (i.e., second generation branching point), which may comprise 2, 3, 4 or 5 branches or polymer arms. Such branched polymer would resemble a second generation dendron. In some embodiments, each of the branches from the second generation branching point may further include an additional branching point (i.e., third generation branching point) comprising 2, 3, 4, or 5 branches, which would from a third generation dendron. In some embodiments, a dendritic arresting construct can have more than one generation of branching points. In some embodiments, a dendritic arresting construct can have 2, 3, 4, or 5 generations.

Examples of branched and dendritic arresting construct moieties include:

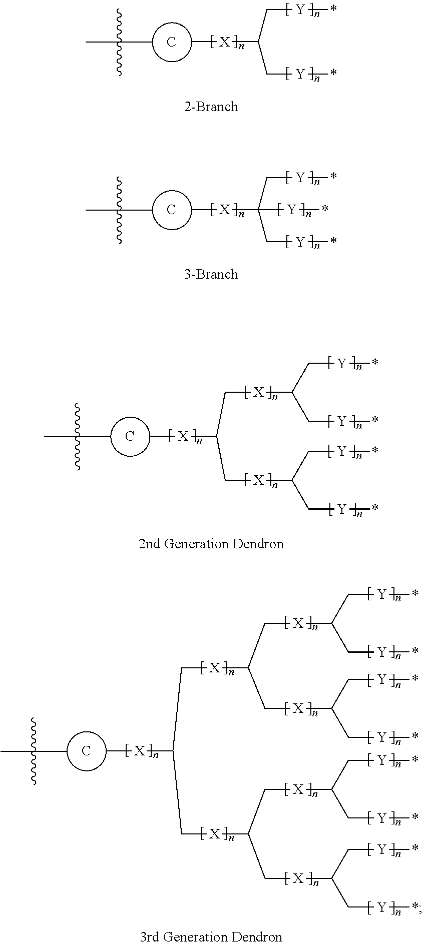

where each of -[X]ₙ- and -[Y]ₙ- represents a polymer selected from synthetic organic polymers, polynucleotides, peptide, or polypeptide sequences (described above), and n refers to the number of repeating units in the polymer,

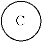

refers to a covalent coupling between the polymer and the nucleotide on the daughter strand, and * is an end group on the polymer chain. The end group on the polymer chain is not particularly limited and may be any proper polymer end group. In some embodiments, * may include, but not limit to, hydrogen, alkyl (e.g., methyl), halogen (e.g., bromide), thiol, amine, dithiobenzoate, dodecyltrithiocarbonate, phenylcarbamodithioate, dimethylacetic acid, hydroxyl, amine, phosphate and phosphorothioate, amide, acetyl, carboxylic acid or amine. In some embodiments, n can range from about 1 to about 10, about 1 to about 20, about 1 to about 30, or about 1 to about 40. In some embodiments,

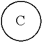

may comprise a coupling moiety selected from the group consisting of amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene. In some embodiments,

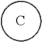

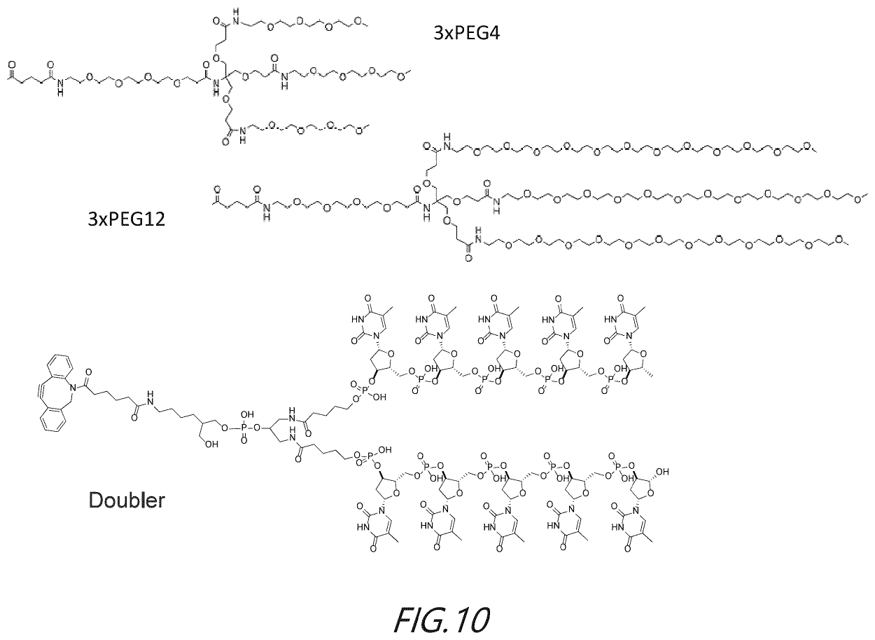

may further comprise a linker between the coupling moiety and the polymer (at X). In some embodiments, [X] and [Y] may be the same. In other embodiments [X] and [Y] may be different. Some examples of arresting constructs comprising branched polymer are shown in FIGS. 10, 11, and 12.

In some embodiments, the arresting construct comprises a cyclic moiety. In some embodiments, the cyclic moiety can include repeating units of organic small molecules, nucleotides, or amino acids. In some embodiments, non-limiting examples of small molecules may include: polyvinyl alcohol, polyvinylpyrrolidone, polystyrene, polystyrene sulfonate, polymethylmethacrylate, polylactic acid, D-glucose, polyethyleneimine, polyacrylamide, glycouril, dialkoxybenezenes, polyethyleneglycol, polycarbonates, polymethymethacrylate or polyamides. In some embodiments, the cyclic moiety can consist of as few as 3 repeating units to as many as 10 repeating units. In some embodiments, the cyclic moiety may have 3, 4, 5, 6, 7, 8, 9, 10, 11, 12, 13, 14, or 15 repeating units. Each of the repeating unit may be a small molecule, a nucleotide, or an amino acid. In some embodiments, the repeating units may be the same. In some embodiments, at least two of the repeating units are different.

Examples of such arresting construct include:

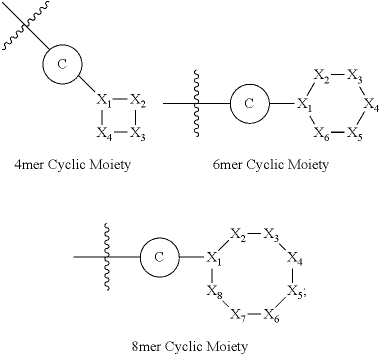

where X₁, X₂, X₃, X₄, X₅, X₆, X₇, X₈ refer to repeat units of small molecules (such as Polyvinyl alcohol, polyvinylpyrrolidone, polystyrene, polystyrene sulfonate, polymethylmethacrylate, polylactic acid, D-glucose, polyethyleneimine, polyacrylamide, glycouril, dialkoxybenezenes, polyethyleneglycol, polycarbonates, polymethymethacrylate or polyamides), nucleotides or amino acids, and

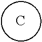

refers to a covalent coupling between the cyclic moiety and the nucleotide on the daughter strand. In some embodiments, X₁, X₂, X₃, X₄, X₅, X₆, X₇, X₈ are the same. In some iterations, X₁, X₂, X₃, X₄, X₅, X₆, X₇, X₈ are distinct groups. In some embodiments,

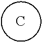

may comprise a coupling moiety selected from the group consisting of amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene. In some embodiments,

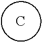

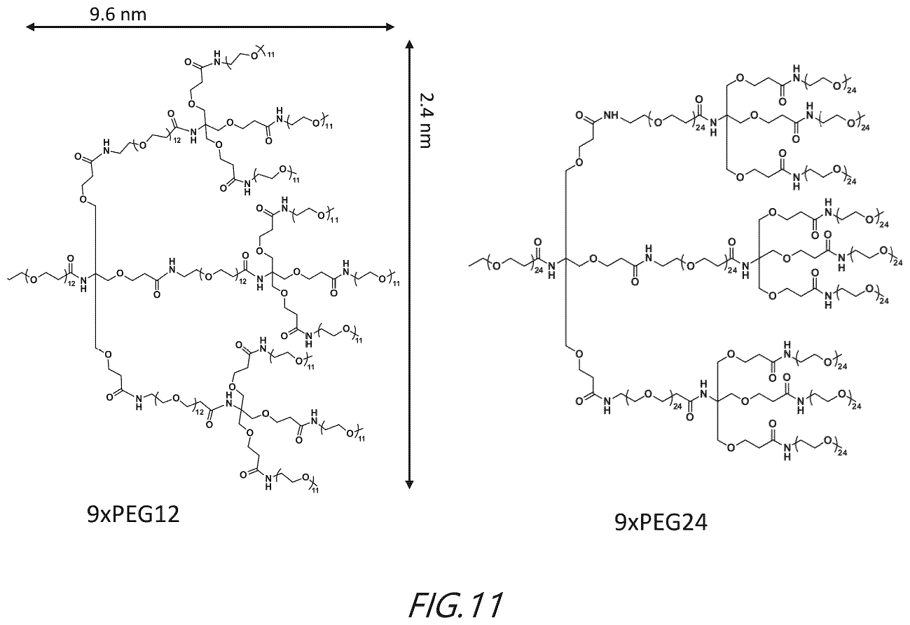

may further comprise a linker between the coupling moiety and the cyclic moiety (at X₁). The cyclic moiety may have 3, 4, 5, 6, 7, 8, 9, 10, 11, 12, 13, 14, or 15 repeating units, thus may include X₁, X₂, X₃, X₄, X₅, X₆, X₇, X₈, X₉, X₁₀, X₁₁, X₁₂, X₁₃, X₁₄, X₁₅. Some examples of arresting constructs comprising branched polymer are shown in FIG. 13.

In some embodiments, use of rigid arresting construct molecules can also improve slowing or stopping capability by restricting conformational changes of the arresting construct in the nanopore system. While cyclic arresting constructs are expected to be less flexible than their linear counterparts, rigidity can be introduced by selection of arresting constructs from a wide range of conjugated chromophores. Non-limiting examples include aromatic small molecules such as perylene, pyrene, rylene diimides, fused heterocylic fluorophores such as Alexa Fluor™ 568 (ThermoFisher Scientific Catalog number A33081), Atto 565 (Atto-Tec Catalog number AD565), Atto 647N (Atto-Tec Catalog number AD647N), porphyrin, phthalocyanine. In some embodiments, rigid arresting constructs can also be chosen from rigid macrocycles. Non-limiting examples include α-, β-, γ-cyclodextrin, cucurbit[n]urils, pillar[n]arenes.

### Non-Covalent Interactions Between Arresting Constructs and Nanopore

While modulation of arresting construct size and geometry is an intuitive and facile approach to improving its arresting ability, increasing arresting construct size indiscriminately is not practical, especially when arresting construct moieties need to be attached at every base of the daughter strand. As arresting construct-modified oligos are synthesized by polymerases (at least in some embodiments) during the library preparation step, engineering polymerases that can incorporate bulky arresting constructs while maintaining polymerase processivity is expected to be a challenge. Moreover, while arresting constructs may help in slowing or arresting translocation, passage of a bulky arresting construct through the pore upon application of a voltage, may “overlap” with adjacent bases resulting in potential loss of information.

Another strategy to increase the ability of an arresting construct to stop or slow the DNA translocation is to enhance the interaction between the arresting construct and the surface of a nanopore. Non-limiting examples of types of interactions, based on charge, polarity, and presence of aromatic residues, include, charge-charge, charge-polar, polar-polar, aromatics-aromatics, non-polar-non-polar. Other permutations and combinations are also contemplated.

For example, non-covalent interaction may include electrostatic interactions, hydrogen-bonding, and hydrophobic interactions. Electrostatic interactions may be present when groups on the arresting construct and the amino acid residues in the internal walls of MspA nanopore are oppositely charged. In some embodiment, hydrogen-bonding can occur via a dipole-dipole attraction between hydrogen bonded to electronegative atom and charged group/long pair. In some embodiments, hydrophobic interactions may be present when alkyl/aromatic groups on the arresting construct associate with hydrophobic residues of the pore in polar solvents.

Amino acid residues on the internal walls of protein pores and surface functional groups on solid state pores provide good opportunities for non-covalent interactions of the arresting construct with the pore surface. In some embodiments, non-covalent interactions can include but are not limited to electrostatic interactions, ion-dipole interactions, dipole-dipole interactions, hydrophobic interactions, or combinations thereof between the arresting construct and surface of nanopores.

In some embodiments, Electrostatic interactions may be present when groups on the arresting construct and the amino acid residues in the internal walls of nanopore are oppositely charged. For example, in the case of protein pores (e.g., MspA), the pore can be engineered to contain specific residues that have electrostatic interactions with the arresting construct moiety, while the arresting construct molecule can be designed to have complementary ionic groups. In some embodiments, the ionic groups on the arresting construct can include but are not limited to negatively charged carboxylate, sulfonate, phosphate functional groups, or combinations thereof. In some embodiments, the ionic groups can also include positively charged primary amines, secondary amines, tertiary amines, quarternary ammoniums, guanidiniums, and combinations thereof.

In some embodiments, arresting constructs can interact via electrostatic attraction between opposite polarities on the arresting construct and the nanopore surface. Without being limited by any particular theory, such interactions are expected to increase residence times of the arresting construct in the constriction. In some embodiments, arresting constructs can also interact with the pore via electrostatic repulsion between similar polarities. Without being limited by any particular theory, such interactions may induce conformational changes in the arresting construct that inhibit its passage through the pore and increase the resident time.

In some embodiments, arresting constructs can also interact with the nanopore surface via ion-dipole interactions. In some embodiments, the arresting construct moiety contains neutral polar groups while the pore surface is functionalized with ionic groups. In other embodiments, the arresting construct moiety contains ionic groups while the pore surface is functionalized with neutral polar groups. In some embodiments, neutral polar functional groups on arresting constructs can include but are not limited to alcohols, thiols, and amides. In some embodiments, polar groups can include, without limitations, fluorinated moieties.

In some embodiments, arresting constructs can also interact with nanopore via dipole-dipole interactions. In some embodiments, the polar residues of the arresting construct and nanopore interact via strong H-bonding. In some embodiments, the polar residues of the arresting construct and nanopore interact via weak Van-der-Waals forces.

Examples of arresting constructs capable of non-covalent interactions with the nanopore include:

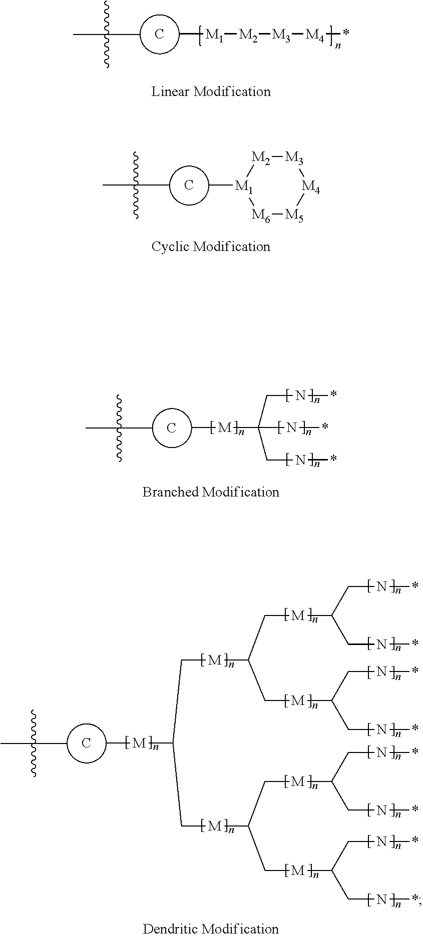

wherein each of M₁, M₂, M₃, M₄, M₅, M₆, M, and N refers to small molecules, nucleotides, or amino acid, n refers to the number of repeating units,

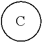

refers to a covalent coupling between the repeating units and the nucleotide on the daughter strand, and * is an end group. The end group on the repeating unit is not particularly limited and may be any proper end group. In some embodiments, * is may include, but not limit to, hydrogen, alkyl (e.g., methyl), halogen (e.g., bromide), thiol, amine, dithiobenzoate, dodecyltrithiocarbonate, phenylcarbamodithioate, dimethylacetic acid, hydroxyl, amine, phosphate, phosphorothioate, amide, acetyl, carboxylic acid or amine. In some embodiments, n can range from about 1 to about 10, about 1 to about 20, about 1 to about 30, or about 1 to about 40. In some embodiments,

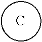

may comprise a coupling moiety selected from the group consisting of amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene. In some embodiments,

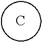

may further comprise a linker between the coupling moiety and M/M₁. In some embodiments, M₁, M₂, M₃, M₄, M₅, M₆ are the same. In other embodiments, M₁, M₂, M₃, M₄, M₅, M₆ are different. In some embodiments, [M] and [N] may be the same. In other embodiments, [M] and [N] may be different. In some embodiments, M₁, M₂, M₃, M₄, M₅, M₆, M and N may be negatively charged. In some embodiments, M₁, M₂, M₃, M₄, M₅, M₆, M and N may be positively charged. In some embodiments, M₁, M₂, M₃, M₄, M₅, M₆, M and N may be neutral polar groups. In some embodiments, M₁, M₂, M₃, M₄, M₅, M₆, M and N may be hydrophobic groups. In some embodiments, M₁, M₂, M₃, M₄, M₅, M₆, M and N may be one or more combinations of negatively charged, positively charged, polar, and hydrophobic groups. In some embodiments, non-limiting examples of small molecules M₁, M₂, M₃, M₄, M₅, M₆, M, and N may include: polyvinyl alcohol, polyvinylpyrrolidone, polystyrene, polystyrene sulfonate, polymethylmethacrylate, polylactic acid, D-glucose, polyethyleneimine, polyacrylamide, glycouril, dialkoxybenezenes, polyethyleneglycol, polycarbonates, polymethymethacrylate or polyamides.

Further example of arresting constructs capable of non-covalent interactions with the nanopore includes a fluoroalkyl moiety.

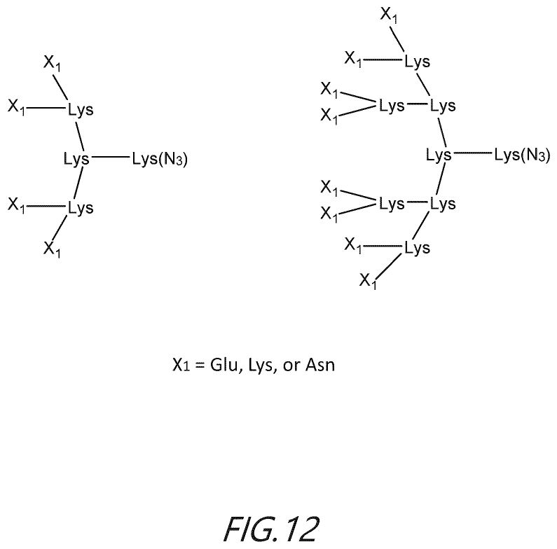

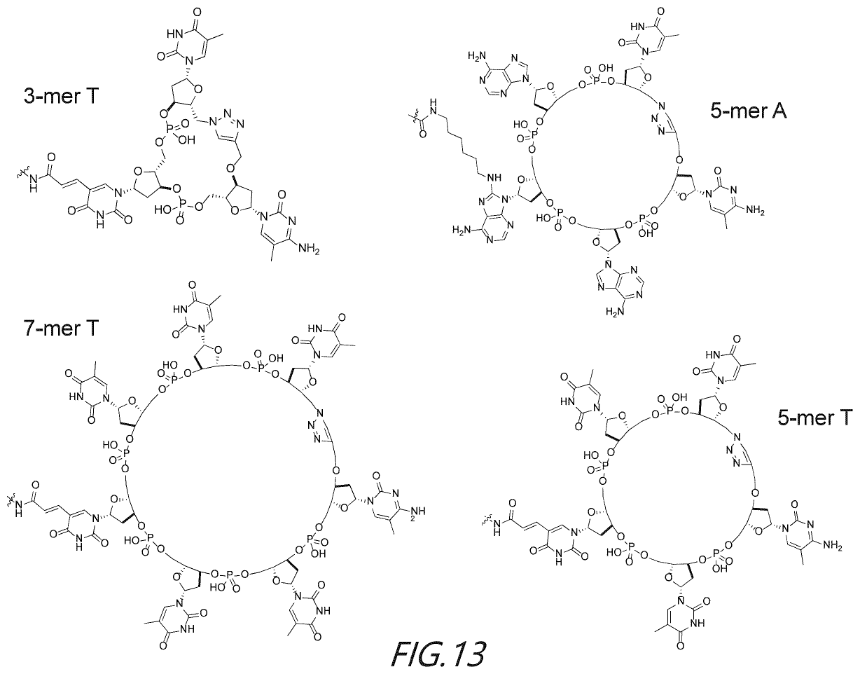

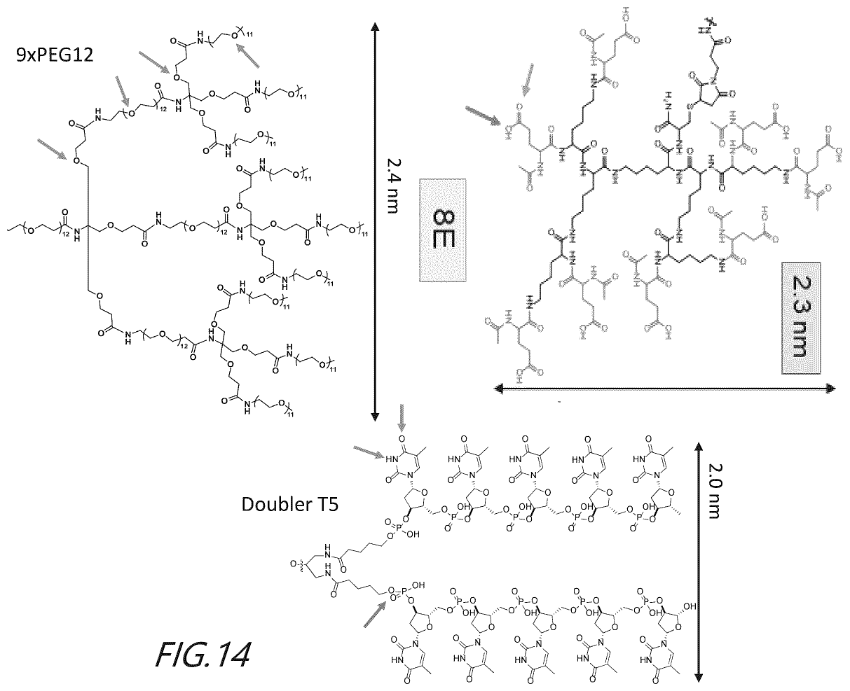

In some embodiments, the ability of an arresting construct to function as one can vary on a number of factors including but not limited to its cross-sectional diameter, charge, and a combination thereof. For example, with reference to FIG. 14, a 9xPEG12 provides a large cross-sectional diameter of about 2.4 nm. A doubler T5 has a smaller cross-sectional diameter of about 2.0 nm as compared to the 9xPEG12 but provides more negative charge. An 8E arresting construct provides both a large cross-sectional diameter of about 2.3 nm as well as more negative charge.

### Examples of Arresting Constructs

In some embodiments, PEG-based arresting constructs are provided. In some embodiments, PEG-based arresting constructs are linear. Non-limiting examples include PEG4, PEG8, PEG12, and PEG24 as shown in FIG. 8. In some embodiments, PEG-based arresting constructs are branched. Non-limiting examples include 3xPEG4, 3xPEG12, 9xPEG12, and 9xPEG24 as shown in FIG. 10 and FIG. 11. For example, a 9xPEG12 arresting construct has a size (in 2-dimensions) of about 9.6 nm×2.4 nm (as determined by, for example, energy minimization modelling using ChemDraw 3D).

In some embodiments, oligo-based arresting constructs are provided. In some embodiments, oligo-based arresting constructs are linear. Non-limiting examples include 3-mer TdT and 5-mer TdTdT as shown in FIGS. 9, and 9-mer. In some embodiments, oligo-based arresting constructs are in the form of a hairpin. In some embodiments, oligo-based arresting constructs are branched. In some embodiments, oligo-based arresting constructs are circular. Non-limiting examples include 3-mer T, 5-mer A, 5-mer T, and 7-mer T as shown in FIG. 13.

In some embodiments, branched peptide-based arresting constructs are provided. In some embodiments, branched peptide-based arresting constructs comprise positive residues (e.g., K), negative residues (e.g., E), polar resides (e.g., N), hydrophobic residues (e.g. W). Non-limiting examples of branched peptide-based arresting constructs include 4K, 4E, 4N, 4W, and 8E shown in FIG. 14. In some embodiments, the branched peptide-based arresting constructs can comprise 2-, 4-, 8-, 2n-branches. In some embodiments, branched peptide-based arresting constructs with negative residues (e.g., E) are preferred. In some embodiments, the size of branched peptide-based arresting constructs (in 2-dimensions) ranges from about 2.1 nm to about 2.6 nm (e.g., 4K, 4E, 4N, and 4W). In some embodiments, the size of branched peptide-based arresting constructs (in 2-dimensions) ranges from about 3.1 nm to about 2.3 nm (e.g., 8E).

In some embodiments, other peptide-based arresting constructs are provided. In some embodiments, other peptide-based arresting constructs include cyclic arresting constructs. Non-limiting examples include Cyclic 6N, Cyclic 6E, Cyclic 6Gla, Cyclic 5E, Cyclic 5Gla, Cyclic 4E, and Cyclic 4Gla. In some embodiments, other peptide-based arresting constructs include branched arresting constructs. Non-limiting examples include 2Gla and 4Gla. In some embodiments, Gla may be preferred over E because Gla provides twice the amount of charge as compared to E. In some embodiments, branched and cyclic arresting constructs are preferred.

In some embodiments, fluoroalkyl-based arresting constructs are provided. Non-limiting examples include F5, F7, F9, and F13. In some embodiments, fluorine in the fluoroalkyl-based arresting constructs interacts with residues in the pore.

### Screening Arresting Constructs

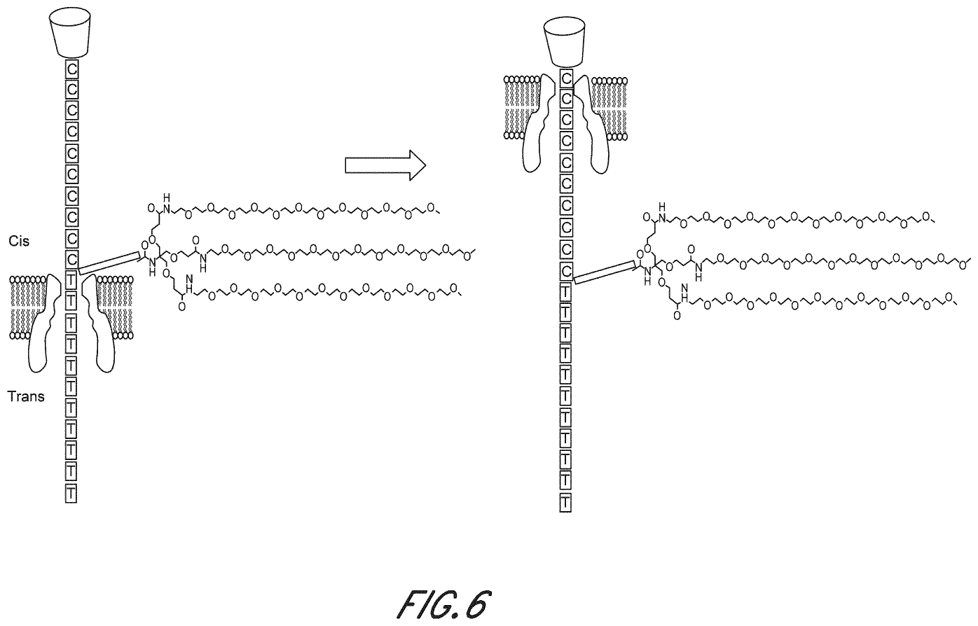

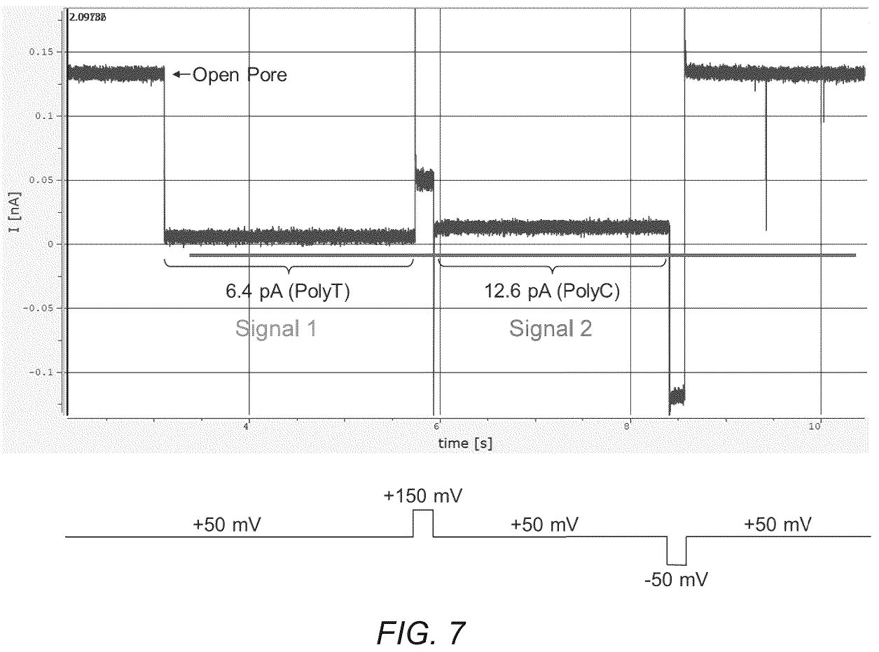

FIG. 6 illustrates an exemplary model system for screening suitable arresting constructs. FIG. 7 shows current-time trace results from an experiment using the model system illustrated in FIG. 6.

FIG. 6 shows that the model system used a branched polyethylene glycol molecule (Catalog No. BP-23454, BroadPharm, San Diego) as the arresting construct moiety to test its stopping ability in a MspA nanopore. The arresting construct was conjugated to the middle of a synthetic oligo of a defined sequence (poly dT followed by poly dC) and added to the cis side of the protein pore. In this model system shown in FIG. 6, which is not drawn to scale as the arresting construct is greatly enlarged, the nanopore is deposited such that the constriction region of the nanopore is closer to the cis side. As shown in FIG. 7, a capture voltage of 50 mV was applied to attract the DNA into the nanopore, and a reading voltage of about 50 mV was constantly applying across the nanopore. FIG. 7 shows that after the arresting construct-modified DNA was captured, a constant current of about 6.4 pA that is characteristic of the DNA bases (poly dT) below the arresting construct was observed. This indicated that the arresting construct had stably arrested the DNA translocation.

Subsequently, a voltage of about 150 mV for 200 msec was applied to force the arresting construct through the nanopore, during which a current signal characteristic of a single-base translocation event was also observed. After the arresting construct passed through the nanopore, translocation resumed until a large protein (Neutravidin) that was attached to the DNA molecule again arrested translocation. FIG. 7 shows that a constant current of about 12.6 pA that is characteristic of bases (poly dC) after the arresting construct passed through was observed. The DNA was ejected from the nanopore by reversing the polarity of the voltage to −50 mV.

### Sequencing Methods

In some embodiments, a method for determining a sequence of a target polynucleotide in a nanopore-based sequencing system is provided.

In some embodiments, the method comprises providing a target polynucleotide, which is a daughter strand of a sample polynucleotide comprising nucleotides with modifications. Each nucleotide is associated with or attached to a modification, the modification comprising one or more arresting constructs and optionally a reporter element or multiple sub-reporter elements. In some embodiments, the daughter strand of the sample polynucleotide comprises nucleotide analogs, wherein the nucleotide analogs comprise chemical structures corresponding to an arbitrary nucleotide of the sample polynucleotide, and wherein the daughter strand is a polymer. In some embodiments, the method comprises applying a capture voltage to cause the daughter strand to insert into the nanopore. A driving voltage is then applied to translocate the target polynucleotide through the nanopore. In some embodiments, the driving voltage is held invariant (e.g. constant) to translocate the target polynucleotide. In some embodiments, the driving voltage may be supplemented with a voltage pulse to translocate the target polynucleotide. The translocation stops or slows when a first modification comprising a first arresting construct interacts with the nanopore, thereby positioning the first nucleotide or the corresponding reporter element in a constriction of the nanopore. The translocation stops or slows again when a second modification comprising a second arresting construct interacts with the nanopore and positioning the second polynucleotide or the corresponding reporter element in the constriction of the nanopore.

In some embodiments of the method, the dwell time of the nucleotide or reporter element in the nanopore is greater than 0.1 ms. In some embodiments of the method, the dwell time of the nucleotide or reporter element in the nanopore is greater than 0.2, 0.3, 0.4, 0.5, 0.6, 0.7, 0.8, 0.9, 1, 2, 3, 4, 5, 6, 7, 8, 9, 10 ms, or any value in between the preceding values. In some embodiments of the method, the dwell time of the nucleotide or reporter element in the nanopore is greater than 10, 15, 20, 25, 30, 35, 40, 45, 50, 55, 60, 65, 70, 75, 80, 85, 90, 95, or 100 ms, or any value in between the preceding values.

In some embodiments of the method, the dwell time of the nucleotide or reporter element in the nanopore is less than 0.1 ms. In some embodiments of the method, the dwell time of the nucleotide or reporter element in the nanopore is less than 0.2, 0.3, 0.4, 0.5, 0.6, 0.7, 0.8, 0.9, 1, 2, 3, 4, 5, 6, 7, 8, 9, 10 ms, or any value in between the preceding values. In some embodiments of the method, the dwell time of the nucleotide or reporter element in the nanopore is less than 10, 15, 20, 25, 30, 35, 40, 45, 50, 55, 60, 65, 70, 75, 80, 85, 90, 95, or 100 ms, or any value in between the preceding values.

The ionic electrical current through the nanopore as the target polynucleotide translocates through the nanopore is recorded, and the changes in the recorded current correlate with the different nucleotide or corresponding reporter element that was in the constriction of the nanopore. The identity of the nucleotide sequence in the target polynucleotide can therefore be determined based on the recorded current trace.

The reporter elements may comprise one or multiple nucleotides in addition to other moieties. In some embodiments, an arresting construct may be attached to the reporter element.

In some embodiments of the method, providing a daughter strand comprises synthesizing a daughter strand using the nucleotides with the modification, and the modification is covalently attached to the nucleotide. In some embodiments of the method, electrical response from the pore depends on the identity of the nucleotide. In some embodiments of the method, electrical response from the pore depends on the identity of the modification attached to each nucleotide. In some embodiments of the method, electrical response from the pore depends on the identity of the reporter element. In some embodiments of the method, electrical response from the pore can be influenced by the spacer in the modification. For example, the distance between adjacent nucleotides, the distance between the adjacent reporter elements or arresting constructs, or the distance between the arresting construct and the reporter element.

In some embodiments of the method, the constriction has an opening with an inner diameter from about 0.6 nm to about 1.2 nm. In some embodiments of the method, the constriction has an opening with an inner diameter from about 0.3 nm to about 2.4 nm. In some embodiments of the method, the constriction has an opening with an inner diameter from about 0.3 nm to about 0.6 nm. In some embodiments of the method, the constriction has an opening with an inner diameter from about 0.6 nm to about 1.2 nm. In some embodiments of the method, the constriction has an opening with an inner diameter from about 1.2 nm to about 1.8 nm. In some embodiments of the method, the constriction has an opening with an inner diameter from about 1.8 nm to about 2.4 nm.

In some embodiments of the method, the arresting construct comprises a polymer selected from the group consisting of a linear synthetic hydrophilic polymer, linear synthetic hydrophobic polymer, linear polynucleotide, linear polypeptide, branched polymer, dendritic polymer, and cyclic polymer. In some embodiments the arresting construct can be configured based upon the inner diameter of the constriction opening. For example, the arresting construct may have a diameter at least about 0.5×, about 1×, about 1.25×, about 1.5×, or about 2× the inner diameter of the constriction opening. For example, FIG. 11 shows that a 9xPEG12 arresting construct has a diameter of 2.4 nm. Thus, if the inner diameter of the constriction opening is about 2.4 nm then the arresting construct is 1× the inner diameter. In some embodiments the inner diameter of the constriction may be about 1.2 nm, which is similar to that of MspA.

In some embodiments of the method, the modification and/or arresting construct comprises a covalent coupling between the polymer and the nucleotide. In some embodiments of the method, the covalent coupling is selected from the group consisting of amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene.

In some embodiments, described herein is a method for determining a sequence of a target polynucleotide in a nanopore-based sequencing system, the method including: providing a target polynucleotide including nucleotides, wherein each nucleotide is attached to a modification configured to slow the target polynucleotide relative to a nanopore; and applying a constant voltage across the nanopore to identify a first reporter element in a constriction of the nanopore based on a first electrical response in the system.

In some embodiments, the modification attached to the nucleotide also translocates through the nanopore. In some embodiments, providing a target polynucleotide includes synthesizing a daughter strand based on a template polynucleotide using nucleotides with modifications, wherein the modifications are covalently attached to the nucleotides. In some embodiments, the target polynucleotide includes at least one additional nucleotide including an additional modification. In some embodiments, at least two of the modifications are the same. In some embodiments, at least two of the modifications are different. In some embodiments, each nucleotide is attached to a modification unique to the type of the nucleotide. In some embodiments, the first electrical response further depends on the modification unique to the nucleotide. In some embodiments, at least one modification includes a cyclic loop. In some embodiments, the cyclic loop further includes an arresting construct, wherein the arresting construct increases the dwell time of the nucleotide in the nanopore. In some embodiments, the modification further comprises one or more spacers. In some embodiments, he modification further comprises one or more reporter elements. In some embodiments, the reporter elements correspond to, and encode for, nucleobase identities. In some embodiments, the one or more spacers distances successive nucleotides and/or arresting constructs.

In some embodiments of the method, the voltage ranges from about 50 mV to about 450 mV. In some embodiments of the method, the voltage ranges from about 75 mV to about 300 mV. In some embodiments of the method, the voltage ranges from about 100 mV to about 200 mV. In some embodiments of the method, the voltage ranges from about 150 mV to about 200 mV. In some embodiments of the method, the voltage ranges from about 200 mV to about 250 mV. In some embodiments of the method, the voltage ranges from about 250 mV to about 400 mV.

In some embodiments of the method, the duration of time of the voltage bias is about 200 msec (millisecond). In some embodiments of the method, the duration of time of the voltage ranges from about 100 msec to about 400 msec. In some embodiments of the method, the duration of time of the voltage ranges from about 100 msec to about 200 msec. In some embodiments of the method, the duration of time of the voltage ranges from about 200 msec to about 300 msec. In some embodiments of the method, the duration of time of the voltage ranges from about 300 msec to about 400 msec. In some embodiments of the method, the duration of the voltage ranges from about 1 μs (microsecond) to about 10 μs, from about 10 μs to about 100 μs, from about 100 μs to about 500 μs, from about 500 μs to about 1000 μs, from about 1 msec to about 100 msec, or any value therebetween.

In some embodiments of the method, the dwell time of the nucleotide in the nanopore is greater than 0.1 ms. In some embodiments of the method, the dwell time of the nucleotide in the nanopore is greater than 0.5 ms.

### Methods of Synthesizing a Target Polynucleotide

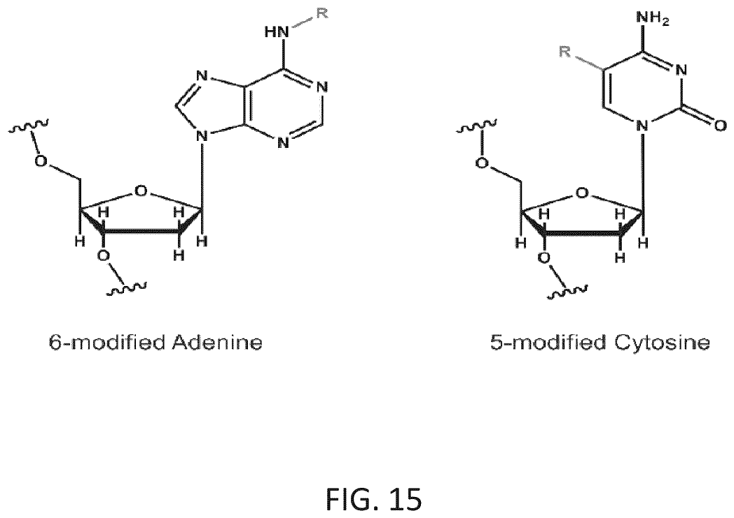

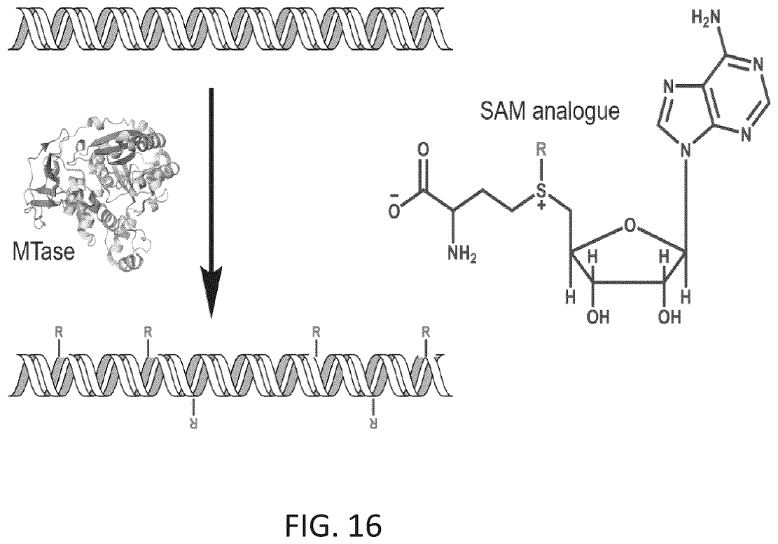

FIG. 15 shows that the exocyclic amine at the sixth position of adenine and the fifth carbon on the cytosine ring are two commonly occurring methylation sites (where the R groups are shown) found in DNA. FIG. 16 illustrates that DNA Methyltransferases (MTases) can catalyze the transfer of methyl groups (the R groups) from S-adenosyl methionine (SAM) analogues to the methylation sites found in a DNA, e.g., the two commonly occurring methylation sites shown in FIG. 15. Based on the process illustrated in FIG. 16, in some embodiments, DNA can be directly modified to have the R groups.

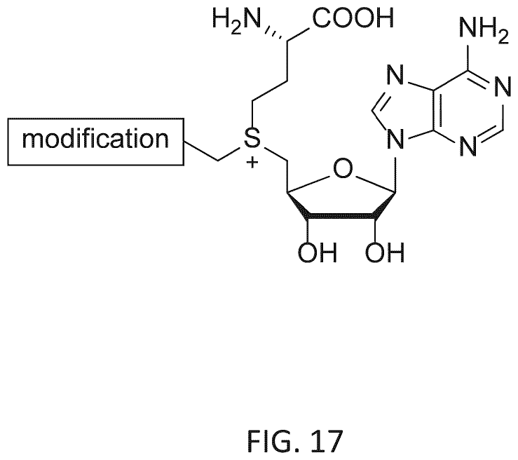

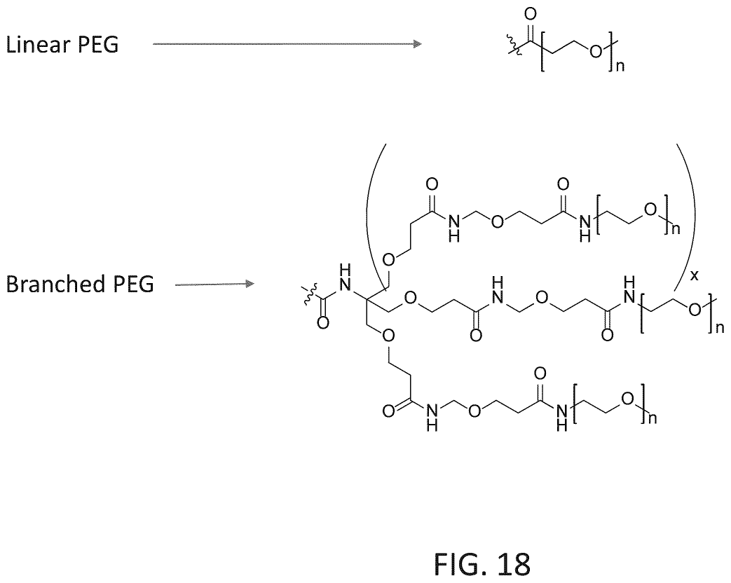

FIG. 17 shows an exemplary structure of a SAM analogue having a modification. In some embodiment, a modification or moiety in a SAM analogue can be transferred to a nucleobase in a ssDNA using a MTase, similar to the process illustrated in FIG. 16. Then, the modified ssDNA can be subject to nanopore sequencing. The modifications or moieties covalently attached to the ssDNA can act as “arresting constructs”, since they can stop the translocation of the ssDNA through a nanopore until an increase in applied voltage drives further translocation. The arresting constructs used as rachets or arresting constructs may include PEG, with different lengths and branching (see FIG. 18), peptides, or oligonucleotides.

Most MTases are sequence dependent, which may limit the potential coverage of modified bases on a DNA molecule. However, in a preferred embodiment, the adenine DNA methyltransferase M.EcoGII may be used. M.EcoGII is sequence independent and is able to methylate up to 99% of adenines in a small dsDNA model substrate. In some embodiments, sequence specific cytosine methyltransferase, such as M.SssI, which recognize CG dinucleotides, may be used. In some embodiments, variants of M.SssI which are engineered to be sequence independent may be used.

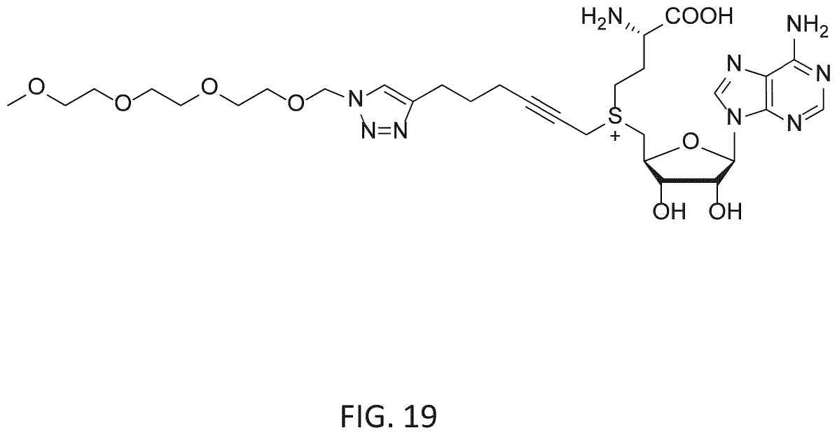

In FIG. 17, an example of the generic structure of a SAM analogue is shown, which includes an arresting construct to be transferred onto an oligonucleotide or polynucleotide. In some embodiments, the arresting construct may be in the form of a linear PEG chain (n=1 to 48), or in the form of a branched PEG (n=1 to 48, x=2 to 9), as shown in FIG. 18. In some embodiments the arresting construct may be a portion of the cyclic loop or a branch off of the cyclic loop. In some embodiments, the PEG chain may be attached to the SAM analogue by covalent couplings such as amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, or azide-norbornene. FIG. 19 shows an example of a SAM analogue having a PEG4 group conjugated via Cu-click reaction. In some embodiments, other possible arresting constructs include fluorophores, oligonucleotides, peptides or any molecules that may be covalently attached to the SAM analogue using one or more of the coupling chemistries listed above.

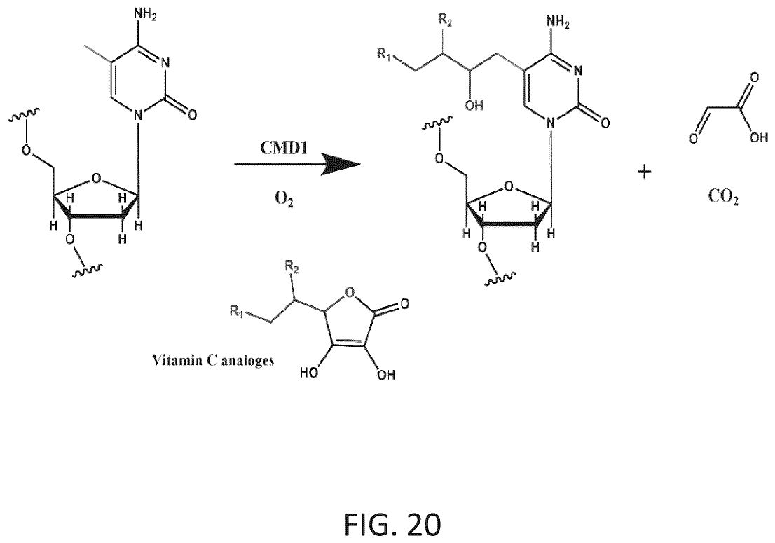

FIG. 20 illustrates an exemplary process of modifying a naturally occurring 5-methylcytosine by using the Ten-Eleven Translocation (TET) dioxygenase homolog CMD1, a 5mC-modifying enzyme found in green alga Chlamydomonas reinhardtii, and an analog of vitamin C. Vitamin C is a natural co-substrate of CMD1. In some embodiments, the vitamin C analog can have modifications (R1 and R2) on the carbon 5 and the carbon 6. In some embodiments, the modifications R1 and R2 may be in the form of a linear PEG chain (n=1 to 48), or in the form of a branched PEG (n=1 to 48, x=2 to 9). Based on the process illustrated in FIG. 20, 5-methylcystosine in a target polynucleotide can be attached with modifications and identified using the disclosed nanopore sequencing methods with controlled polynucleotide translocation.

### Cyclic Loop Modifications

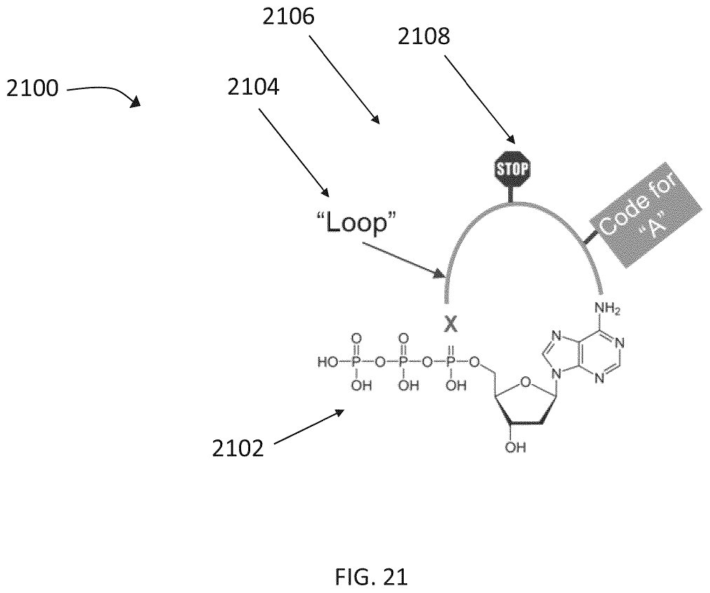

FIG. 21 illustrates a generic embodiment of a modified nucleotide that can be used to form a daughter strand of the sample polynucleotide. The modified nucleotide has a cyclic loop modification 2104 appended to the nucleotide 2102 at two locations, thereby forming a cyclic loop. The cyclic loop modification further includes an arresting construct 2106 configured to slow or modulate the translocation speed of the target polynucleotide through a nanopore readhead. The cyclic loop modification further includes a reporter element 2108 encoding each of the nucleobases. Each reporter element 2108 produces a specific signal when traversing the nanopore, and the reporter element 2108 for each nucleobase produce substantially different signals. These signals are differentiated within the range of voltages used in the method disclosed herein.

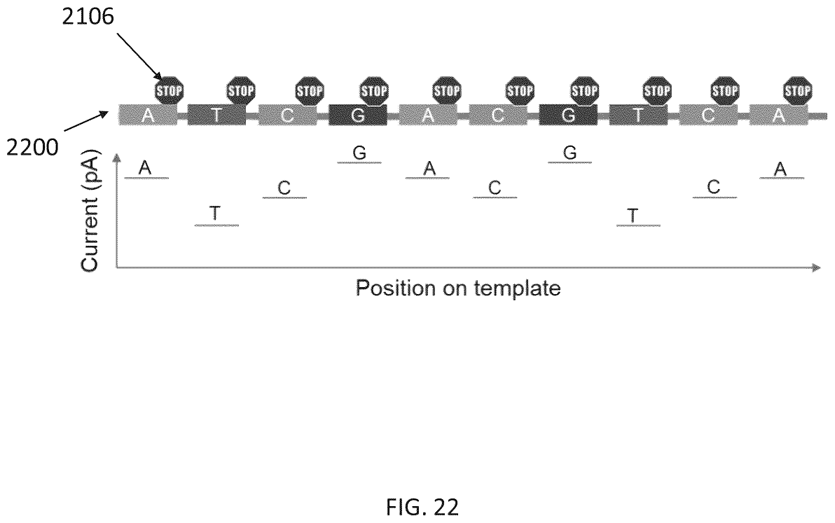

FIG. 22 illustrates a simplified depiction of an elongated polynucleotide 2200 comprising arresting constructs 2106 and reporter elements (represented by A, T, C, G, etc.), wherein each reporter element produces a distinguishable signal when traversing a nanopore. In some embodiments, cyclic loop modifications are incorporated into daughter strands. In some embodiments, each cyclic loop nucleotide contains a unique barcoding/reporter region (i.e., reporter element) that is specific to the original bases (e.g., A, T, C, or G) and a cleavable site. The daughter strand is then “elongated” by cutting the cleavable sites. Consequently, when sequencing the daughter strand, the nanopore can “read” the barcoding/reporter region to identify the base that it is coding for. The reporter element that is introduced in the daughter strand via polymerization is designed to occupy the readhead of the nanopore entirely with a spacer that increases the distance between the adjacent reporter elements, hence reducing the number of signals to just four, i.e., one per nucleobase. Thus, the disclosed technology allows barcode-based decoding of individual bases.

In some embodiments, an arresting construct may be present to modulate translocation speed of the elongated cyclic loop polynucleotide. In some embodiments, the cyclic loops contain non-barcoding spacer which allow the daughter strand to elongate after cutting the cleavable sites. The non-barcoding spacer constructs may produce a distinguishable signal or a distinguishable signal break from signals of the nucleobases when passing through the nanopore, thereby isolating and/or enhance the recorded signals from the nucleobases. Thus, the disclosed technology allows improved resolution of the recorded signal. In some embodiments, the spacer may contain both barcoding/reporter regions and non-barcoding spacer constructs. In some embodiments, the spacer construct may be the barcoding/reporter clement.

### Base Calling

Synthetic cyclic loop constructs were assessed for translocation of constituent nucleotides across a nanopore according to Table 1, with sequence abbreviations detailed in Table 2. All constructs were incubated with Traptavidin 1:1 in KCI buffer prior to sequencing. A polymer membrane (5 mg/mL P5 in octanc) was formed across a 50 μm aperture, separating the cis and trans compartments. While the membrane traversed symmetric buffer conditions (1M KCl, 50 mM Hepes pH 7.5), only the trans compartment contained “Lock Sh16LNA” (5 uM). M2NNN MspA was inserted into the membranes and the ionic current through the nanopore was recorded at a 10 kHz sampling rate.

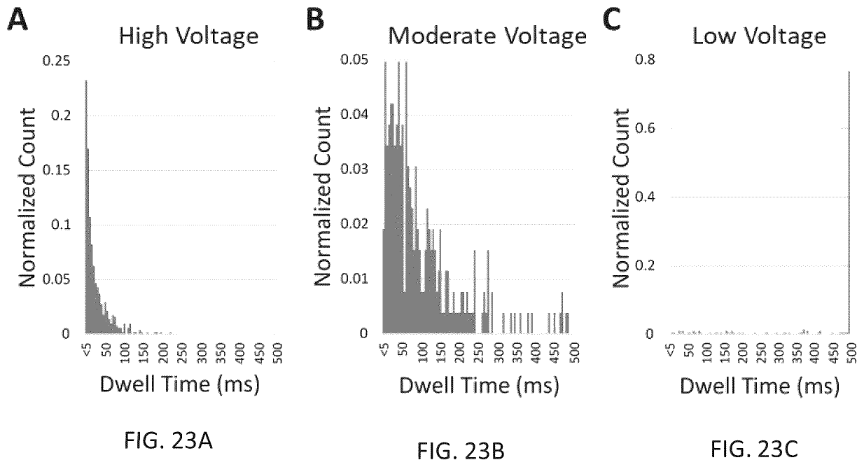

FIGS. 23A-23C illustrate the distribution of dwell times for a single arresting construct, using Construct 1 from Table 1. As shown in FIG. 23A, at a high driving voltage, dwell times were fit to an exponential decay. In FIG. 23B, dwell times were fit closely to a gamma distribution when held at a moderate voltage. In this example, approximately 2% of events showed a duration <5 ms. In contrast, FIG. 23C illustrates that at a low positive voltage, the arresting construct was immobilized in the pore until an arbitrary experimental cutoff at 500 ms.

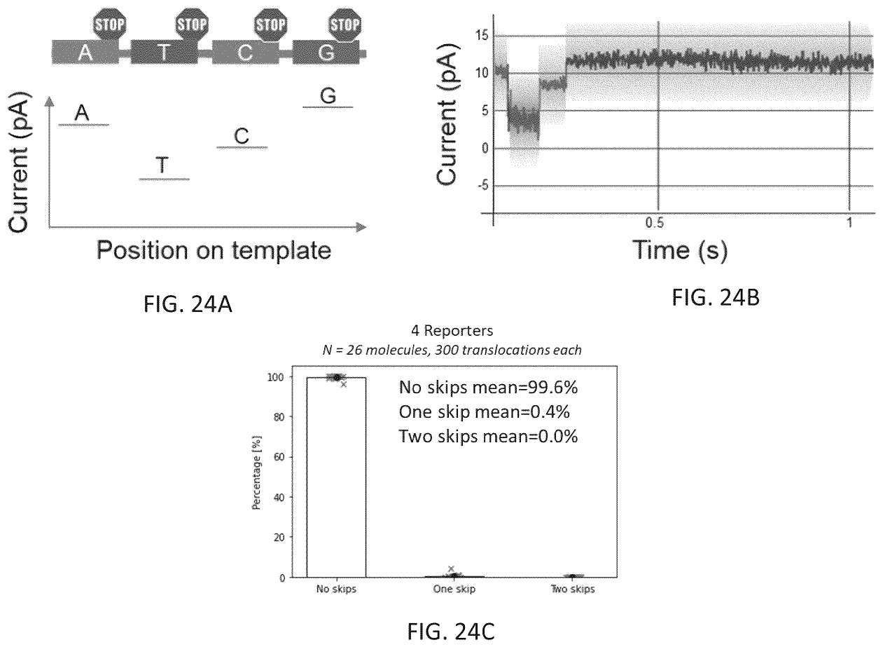

FIG. 24A-24C relate to current traces and statistics for a synthetic construct derived from Construct 1 from Table 1. Specifically, FIG. 24A illustrates a heterogenous sequence of 4 elongated cyclic nucleotides from Construct 1. FIG. 24B illustrates a current trace from the synthetic construct containing 4 arresting constructs and 4 reporter elements corresponding to A, T, C, and G. FIG. 24C illustrates the statistical distribution for 26 translocated molecules, indicating 99.6% of translocation events detected for all 4 reporters.

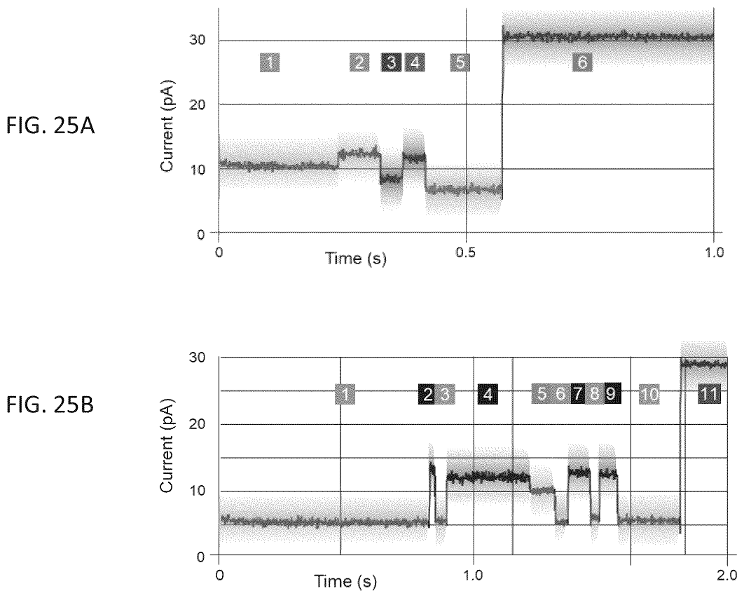

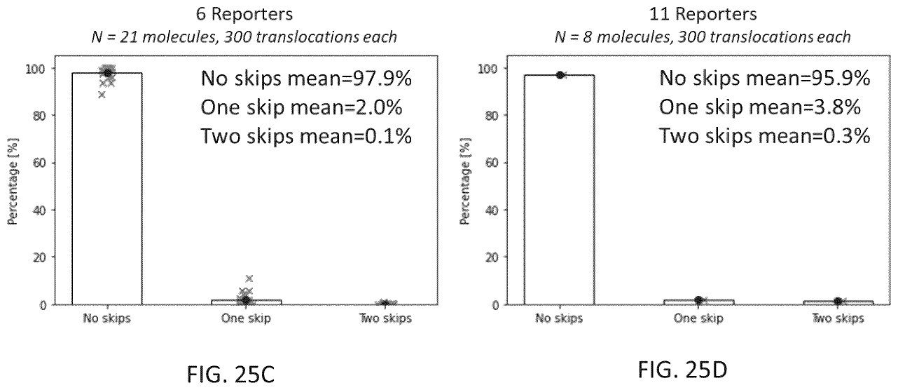

FIG. 25A-25D illustrates current traces and statistics for a synthetic construct derived from various constructs described in Table 1. FIG. 25A describes a current trace of a synthetic construct derived from Construct 2 in Table 1, containing 6 arresting constructs and 6 reporter elements. FIG. 25B illustrates a current trace from a synthetic construct derived from Construct 3 in Table 1, containing 11 reporter elements. As can be appreciated from FIGS. 25A and 25B, 6 and 11 distinct signals were observed for each reporter element. FIGS. 25C and 25D illustrate aggregated statistics for the 6- and 11-reporter elements from FIGS. 25A and 25B respectively, showing >95% of translocation events detected for all reporter elements.

TABLE 1
Cyclic Loop Constructs

| Construct | Construct | |
|---|---|---|
| # | Name | Construct Sequence (5′ to 3′) |
| 1 | 3-ARC | /5Phos/ TTTCGCGCAGACTCCGCCCCCCCCT /iSp18//iSp18/ (SEQ |
| | 2xSp18 + | ID NO: 1) |
| | B4E | AGAGAA /dT-B4E/ ACCCCC /iSp18//iSp18/ TTTTTT /dT-B4E/ |
| | | TCCCCC /iSp18//iSp18/ CTCCCT /dT-B4E/ ACCCCC /iSp18/ |
| | | /iSp18/ GAACGGCGAGCAGAG /3Bio/ (SEQ ID NO: 2) |
| 2 | 5-ARC | /5Phos/ TTTCGCGCAGACTCCGCCCCCCCCT /iSp18//iSp18/ (SEQ |
| | 2xSp18 + | ID NO: 3) |
| | B4E | CTCCCT /dT-B4E/ ACCCCT /iSp18//iSp18/ AGAGAA /dT-B4E/ |
| | | ACCCCC /iSp18//iSp18/ TTCTTG /dT-B4E/ ACCCCC /iSp18//iSp18/ |
| | | ATAGAC /dT-B4E/ TCCCCC /iSp18//iSp18/ TTTTTT /dT-B4E/ |
| | | TCCCCC /iSp18/ |
| | | /iSp18/ GAACGGCGAGCAGAG /3Bio/ (SEQ ID NO: 4) |
| 3 | 10-ARC | /5Phos/ TTTCGCGCAGACTCCGCCCCCCCCT /iSp18//iSp18/ (SEQ |
| | 2xSp18 + | ID NO: 5) |
| | B4E | TTTTTT /dT-B4E/ TCCCCT /iSp18//iSp18/ AGAGAA /dT-B4E/ |
| | | ACCCCC /iSp18//iSp18/ TTCTTG /dT-B4E/ ACCCCC /iSp18//iSp18/ |
| | | ATAGAC /dT-B4E/ TCCCCC /iSp18//iSp18/ CTCCCT /dT-B4E/ |
| | | ACCTGC /iSp18//iSp18/ AGAGAA /dT-B4E/ ATTTTT /iSp18//iSp18/ |
| | | TTTTTT /dT-B4E/ TTTCCC /iSp18//iSp18/ ATAGAC /dT-B4E/ |
| | | TTTTTT /iSp18//iSp18/ TTCTTG /dT-B4E/ ATTTTT /iSp18//iSp18/ |
| | | TTTTTT /dT-B4E/ TCCCCC /iSp18/ |
| | | /iSp18/ GAACGGCGAGCAGAG /3Bio/ (SEQ ID NO: 6) |
| 4 | 3-ARC | /5Phos/ |
| | 1xSp18 + | TTTTTTTTTGACTCCCGCGCAGACTCCGCCCTATTTTCCCCAGAA |
| | B4E | /dT-B4E/ (SEQ ID NO: 7) |
| | | CTCTC /Sp18/ AAGTG /dT-B4E/ CGAAG /Sp18/ GAAGT /dT-B4E/ |
| | | CCCC |
| | | /Sp18/ TGTGTTTTTTTTTTTTTT /3Bio/ (SEQ ID NO: 8) |
| 5 | 3-ARC | /5Phos/ TTTTTTTTTCGCGCAGACTCCGCCTTTTCCCCAGAA /dT- |
| | 4xSp18 + | B4E/ (SEQ ID NO: 9) |
| | B4E | CTCTCC/Sp18//Sp18/ TT/Sp18//Sp18/ GAAGTG /dT-B4E/ CGAAGA |
| | | /Sp18//Sp18/ TT /Sp18//Sp18/ AGAAGT /dT-B4E/ CCCC |
| | | /Sp18/ TGTGTTTTTTTTTTTTTT /3Bio/ (SEQ ID NO: 10) |
| 6 | 3-ARC | /5Phos/ |
| | 8xSp18 + | TTTTTTTTTGACTCCCGCGCAGACTCCGCCCTATTTTCCCCAGAA |
| | B4E | /dT-B4E/ (SEQ ID NO: 11) |
| | | CTCTCC /Sp18//Sp18/ TT /Sp18//Sp18/ TT /Sp18//Sp18/ TT |
| | | /Sp18//Sp18/ GAAGTG /dT-B4E/ CGAAGA /Sp18//Sp18/ TT |
| | | /Sp18//Sp18/ TT /Sp18//Sp18/ TT /Sp18//Sp18/ AGAAGT /dT-B4E/ |
| | | CCCC |
| | | /Sp18/ TGTGTTTTTTTTTTTTTT /3Bio/ (SEQ ID NO: 12) |
| 7 | 5-ARC | /5Phos/ TTTCGCGCAGACTCCGCCCCCCCCT /iSp18//iSp18/ (SEQ |
| | BC1,5 + | ID NO: 13) |
| | B4E | TTTTTT /dT-B4E/ TCCCCT /iSp18//iSp18/ AGAGAA /dT-B4E/ |
| | | ACCCCC /iSp18//iSp18/ TTTTTT /dT-B4E/ TCCCCC /iSp18//iSp18/ |
| | | AGAGAA /dT-B4E/ ACCCCC /iSp18//iSp18/ TTTTTT /dT-B4E/ |
| | | TCCCCC /iSp18/ |
| | | /iSp18/ GAACGGCGAGCAGAG /3Bio/ (SEQ ID NO: 14) |
| 8 | 10-ARC | /5Phos/ TTTCGCGCAGACTCCGCCCCCCCCT /iSp18//iSp18/ (SEQ |
| | BC1,5 + | ID NO: 15) |
| | B4E | TTTTTT /dT-B4E/ TCCCCT /iSp18//iSp18/ AGAGAA /dT-B4E/ |
| | | ACCCCC /iSp18//iSp18/ TTTTTT /dT-B4E/ TCCCCC /iSp18//iSp18/ |
| | | AGAGAA /dT-B4E/ ACCCCC /iSp18//iSp18/ CTCCCT /dT-B4E/ |
| | | ACCTGC /Sp18//Sp18/ TTTTTT /dT-B4E/ TTTTTT /Sp18//Sp18/ |
| | | AGAGAA /dT-B4E/ ATTTCC /Sp18//Sp18/ TTTTTT /dT-B4E/ TTTTTT |
| | | /Sp18//Sp18/ AGAGAA /dT-B4E/ ATCTTT /Sp18//Sp18/ TTTTTT /dT- |
| | | B4E/ TCCCCC /Sp18/ |
| | | /Sp18/ GAACGGCGAGCAGAG /3Bio/ (SEQ ID NO: 16) |
| 9 | Lock | + G + G + G + C + G + G + AGTCTGCGCG (SEQ ID NO: 17) |
| | Sh16LNA | |

Definition for Abbreviations in Table 1:

| | |
|---|---|
| /iSp18/ | 18-atom hexa-ethyleneglycol spacer |
| /5Phos/ | 5′ Phosphorylation |
| /3Bio/ | 3′ Biotin |
| /dT-B4E/ | Arresting construct: alkyne dT phosphoramidite |
| | with branched Glutamic acid (×4) |
| + | LNA subunit |

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 nm to about 20 nm should be interpreted to include not only the explicitly recited limits of from about 2 nm to about 20 nm, but also to include individual values, such as about 3.5 nm, about 8 nm, about 18.2 nm, etc., and sub-ranges, such as from about 5 nm to about 10 nm, etc. Furthermore, when “about” and/or “substantially” are/is utilized to describe a value, this is meant to encompass minor variations (up to +/−10%) from the stated value.

While several examples have been described in detail, it is to be understood that the disclosed examples may be modified. Therefore, the foregoing description is to be considered non-limiting.

While certain examples have been described, these examples have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the components for an energy storage system described herein can be provided separately, or integrated together (e.g., packaged together, or attached together) to form an energy storage system.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Although the foregoing invention has been described in terms of certain preferred embodiments, other embodiments will be apparent to those of ordinary skill in the art. Additionally, other combinations, omissions, substitutions and modification will be apparent to the skilled artisan, in view of the disclosure herein. Accordingly, the present invention is not intended to be limited by the recitation of the preferred embodiments, but is instead to be defined by reference to the appended claims. All references cited herein are incorporated by reference in their entirety.

The terminology used in the description presented herein is not intended to be interpreted in any limited or restrictive manner and unless otherwise indicated refers to the ordinary meaning as would be understood by one of ordinary skill in the art in view of the specification. Furthermore, embodiments may comprise, consist of, consist essentially of, several novel features, no single one of which is solely responsible for its desirable attributes or is believed to be essential to practicing the embodiments herein described. As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

Although this disclosure is in the context of certain embodiments and examples, those of ordinary skill in the art will understand that the present disclosure extends beyond the specifically disclosed embodiments to other alternative embodiments and/or uses of the embodiments and obvious modifications and equivalents thereof. In addition, while several variations of the embodiments have been shown and described in detail, other modifications, which are within the scope of this disclosure, will be readily apparent to those of ordinary skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments of the disclosure. Thus, it is intended that the scope of the present disclosure herein disclosed should not be limited by the particular disclosed embodiments described above.

What is claimed is:

1. A method for determining a sequence of a target polynucleotide with a nanopore-based sequencing system, the method comprising:
providing a target polynucleotide comprising nucleotides, wherein each nucleotide comprises a modification, wherein the modification comprises an arresting construct configured to arrest translocation of the target polynucleotide through a nanopore;
applying a driving voltage to translocate one or more portions of the target polynucleotide through a nanopore;
measuring a current of the nanopore continuously during translocation; and
identifying the sequence of the target polynucleotide by correlating the measured current to an identity of one or more nucleotides.

2. The method of claim 1, wherein providing the target polynucleotide comprises synthesizing a daughter strand based on a template polynucleotide using nucleotides with modifications, wherein the modifications are covalently attached to the nucleotides.

3. The method of any one of claim 1, wherein the modification further comprises a cyclic loop, wherein the arresting construct is attached to the cyclic loop.

4. The method of claim 3, wherein the cyclic loop further comprises a reporter element that encodes the nucleotide.

5. The method of claim 4, wherein the cyclic loop further comprises a spacer.

6. The method of claim 1, wherein providing the target polynucleotide comprises:
synthesizing a daughter strand based on a template polynucleotide using nucleotides with modifications, wherein the modifications are covalently attached to the nucleotides; and
cleaving the daughter strand to generate the target polynucleotide having an elongated polynucleotide strand.

7. The method of claim 1, wherein the nucleotide has a dwell time in the nanopore of greater than 5 ms.

8. The method of claim 1, wherein the nucleotide has a dwell time in the nanopore of greater than 0.1 ms.

9. The method of claim 1, wherein the driving voltage is kept constant during the translocation.

10. The method of claim 4, wherein the measured current is dependent on the reporter element or the nucleotide passing through the nanopore.

11. The method of claim 1, wherein the arresting construct comprises a polymer selected from the group consisting of a linear synthetic hydrophilic polymer, linear synthetic hydrophobic polymer, linear polynucleotide, linear polypeptide, branched polymer, dendritic polymer, cyclic polymer, fluoroalkyl, rigid conjugated chromophores, and rigid macrocycles.

12. The method of claim 11, wherein the arresting construct comprises a covalent coupling between the polymer and the corresponding nucleotide or a cyclic loop.

13. The method of claim 12, wherein the covalent coupling is selected from the group consisting of amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene.

14. The method of claim 11, wherein the linear synthetic hydrophilic polymer is selected from the group consisting of polyethyleneglycol, polyvinylalcohol, polyacrylamide, polyvinylpyrrolidone, polystyrenesulfonate, polyethyleneimine, and a combination thereof.

15. The method of claim 11, wherein the linear synthetic hydrophobic polymer is selected from the group consisting of polylactic acid, polymethymethacrylate, polystyrene, and a combination thereof.

16. The method of claim 11, wherein the linear polynucleotide is a homopolymer of a natural nucleotide, a homopolymer of an unnatural nucleotide, a mixed sequence polymer of natural nucleotides, or a mixed sequence polymer of unnatural nucleotides.

17. The method of claims 11, wherein the linear polypeptide comprises one or more types of amino acids.

18. The method of claim 11, wherein the branched polymer comprises 2 or more branches.

19. The method of any one of claims 11, wherein the cyclic polymer has 3 or more repeating units.

20. The method of claim 19, each repeating unit is a small molecule, a nucleotide, or an amino acid.

21. The method of claim 19, wherein the repeating units are the same.

22. The method of claim 19, wherein at least two of the repeating units are different.

23. The method of claim 1, wherein the arresting construct interacts with the nanopore via a non-covalent interaction.

24. The method of claim 23, wherein the non-covalent interaction comprises electrostatic interactions, ion-dipole interactions, dipole-dipole interactions, hydrophobic interactions, and combinations thereof.

25. A system for determining a sequence of a target polynucleotide using a method according to claim 1.

26. A cyclic loop nucleotide comprising a cyclic loop modification bridging a nucleobase and a phosphate group, wherein the cyclic loop modification comprises a reporter encoding the identity of the nucleobase, and an arresting construct adjacent to the reporter.

27. The cyclic loop nucleotide of claim 26, wherein the arresting construct is adjacent to the reporter.

28. The cyclic loop nucleotide of claim 26, wherein the arresting construct comprises a linear, branched, cyclic, or dendritic structure.

29. The cyclic loop nucleotide of claim 26, wherein the cyclic loop nucleotide has one of the following structures: 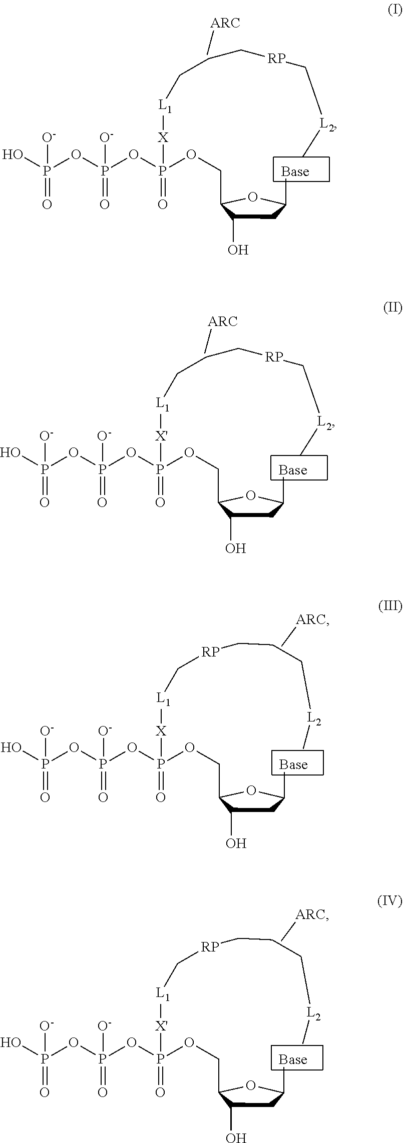
wherein:
X is —O—, —CH₂—, —NSO₂—, —NH—, 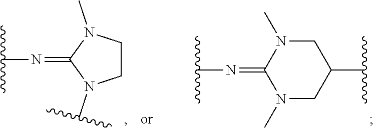
X′ is —S—, ═N—SO₂—; ═NH—CO—, or 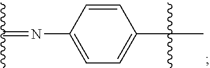 [SMILES: *c1ccc(N=*)cc1]
Base is the nucleobase;
L₁ and L₂ are each a linking group;
RP is the reporter encoding the nucleobase;
and ARC is the arresting construct.

30. The cyclic loop nucleotide of claim 29, wherein the ARC is covalently attached to the cyclic loop via a covalent coupling selected from amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene.

31. A kit for performing a method for determining a sequence of a target polynucleotide in a nanopore-based sequencing system, the kit comprising one or more cyclic loop nucleotides according to claim 26.


SEQUENCE LISTING (17 sequences)

SEQ ID NO: 1 - DNA, 25 bp, synthetic construct
  misc_feature 1..25: note = Synthetic Oligonucleotide
  source 1..25: mol_type = other DNA
  misc_feature 1: note = 5' end is Phosphorylated
  misc_feature 25: note = 3' hydroxy end is linked to 18-atom hexa-ethyleneglycol spacer
tttcgcgcagactccgcccccccct

SEQ ID NO: 2 - DNA, 15 bp, synthetic construct
  misc_feature 1..15: note = Synthetic Oligonucleotide
  source 1..15: mol_type = other DNA
  misc_feature 1: note = 5' end is linked to 18-atom hexa-ethyleneglycol spacer
  misc_feature 15: note = 3' end is Biotintylated
gaacggcgagcagag

SEQ ID NO: 3 - DNA, 25 bp, synthetic construct
  misc_feature 1..25: note = Synthetic Oligonucleotide
  source 1..25: mol_type = other DNA
  misc_feature 1: note = 5' end is Phosphorylated
  misc_feature 25: note = 3' hydroxy end is linked to 18-atom hexa-ethyleneglycol spacer
tttcgcgcagactccgcccccccct

SEQ ID NO: 4 - DNA, 15 bp, synthetic construct
  misc_feature 1..15: note = Synthetic Oligonucleotide
  source 1..15: mol_type = other DNA
  misc_feature 1: note = 5' end is linked to 18-atom hexa-ethyleneglycol spacer
  misc_feature 15: note = 3' end is Biotintylated
gaacggcgagcagag

SEQ ID NO: 5 - DNA, 25 bp, synthetic construct
  misc_feature 1..25: note = Synthetic Oligonucleotide
  source 1..25: mol_type = other DNA
  misc_feature 1: note = 5' end is Phosphorylated
  misc_feature 25: note = 3' hydroxy end is linked to 18-atom hexa-ethyleneglycol spacer
tttcgcgcagactccgcccccccct

SEQ ID NO: 6 - DNA, 15 bp, synthetic construct
  misc_feature 1..15: note = Synthetic Oligonucleotide
  source 1..15: mol_type = other DNA
  misc_feature 1: note = 5' end is linked to 18-atom hexa-ethyleneglycol spacer
  misc_feature 15: note = 3' end is Biotintylated
gaacggcgagcagag

SEQ ID NO: 7 - DNA, 45 bp, synthetic construct
  source 1..45: mol_type = other DNA
  misc_feature 1..45: note = Synthetic Oligonucleotide
  misc_feature 1: note = 5' end is Phosphorylated
  misc_feature 45: note = 3' hydroxy end is linked to alkyne dT phosphoramidite with branched Glutamic acid (x4)
tttttttttgactcccgcgcagactccgccctattttccccagaa

SEQ ID NO: 8 - DNA, 18 bp, synthetic construct
  source 1..18: mol_type = other DNA
  misc_feature 1..18: note = Synthetic Oligonucleotide
  misc_feature 1: note = 5' end is linked to 18-atom hexa-ethyleneglycol spacer
  misc_feature 18: note = 3' end is Biotintylated
tgtgtttttttttttttt

SEQ ID NO: 9 - DNA, 36 bp, synthetic construct
  source 1..36: mol_type = other DNA
  misc_feature 1..36: note = Synthetic Oligonucleotide
  misc_feature 1: note = 5' end is Phosphorylated
  misc_feature 36: note = 3' hydroxy end is linked to alkyne dT phosphoramidite with branched Glutamic acid (x4)
tttttttttcgcgcagactccgccttttccccagaa

SEQ ID NO: 10 - DNA, 18 bp, synthetic construct
  source 1..18: mol_type = other DNA
  misc_feature 1..18: note = Synthetic Oligonucleotide
  misc_feature 1: note = 5' end is linked to 18-atom hexa-ethyleneglycol spacer
  misc_difference 18: note = 3' end is Biotintylated
tgtgtttttttttttttt

SEQ ID NO: 11 - DNA, 45 bp, synthetic construct; identical to SEQ ID NO: 7
  source 1..45: mol_type = other DNA
  misc_feature 1..45: note = Synthetic Oligonucleotide

SEQ ID NO: 12 - DNA, 18 bp, synthetic construct
  source 1..18: mol_type = other DNA
  misc_feature 1..18: note = Synthetic Oligonucleotide
  misc_feature 1: note = 5' end is linked to 18-atom hexa-ethyleneglycol spacer
  misc_feature 18: note = 3' end is Biotintylated
tgtgtttttttttttttt

SEQ ID NO: 13 - DNA, 25 bp, synthetic construct
  source 1..25: mol_type = other DNA
  misc_feature 1..25: note = Synthetic Oligonucleotide
  misc_feature 1: note = 5' end is Phosphorylated
  misc_feature 25: note = 3' end is linked to 18-atom hexa-ethyleneglycol spacer
tttcgcgcagactccgcccccccct

SEQ ID NO: 14 - DNA, 15 bp, synthetic construct
  source 1..15: mol_type = other DNA
  misc_feature 1..15: note = Synthetic Oligonucleotide
  misc_feature 1: note = 5' end is linked to 18-atom hexa-ethyleneglycol spacer
  misc_feature 15: note = 3'end is Biotintylated
gaacggcgagcagag

SEQ ID NO: 15 - DNA, 25 bp, synthetic construct
  source 1..25: mol_type = other DNA
  misc_feature 1..25: note = Synthetic Oligonucleotide
  misc_feature 1: note = 5' end is Phosphorylated
  misc_feature 25: note = 3' end is linked to 18-atom hexa-ethyleneglycol spacer
tttcgcgcagactccgcccccccct

SEQ ID NO: 16 - DNA, 15 bp, synthetic construct
  source 1..15: mol_type = other DNA
  misc_feature 1..15: note = Synthetic Oligonucleotide
  misc_feature 1: note = 5' end is linked to 18-atom hexa-ethyleneglycol spacer
  misc_feature 15: note = 3' end is Biotintylated
gaacggcgagcagag

SEQ ID NO: 17 - DNA, 16 bp, synthetic construct
  source 1..16: mol_type = other DNA
  modified_base 1..7: mod_base = OTHER; note = LNA subunit
gggcggagtctgcgcg